(19) United States
(12) Patent Application Publication
(10) Pub. No.: US 2023/0248772 A1
(43) Pub. Date: Aug. 10, 2023

(54) PRODUCTS AND METHODS USING A PLATELET-DERIVED HEMOSTATIC AGENT FOR CONTROLLING BLEEDING AND IMPROVING HEALING

(71) Applicant: Cellphire, Inc., Rockville, MD (US)

(72) Inventor: Stephen H. Willard, Chevy Chase, MD (US)

(73) Assignee: Cellphire, Inc., Rockville, MD (US)

(21) Appl. No.: 18/301,946

(22) Filed: Apr. 17, 2023

Related U.S. Application Data
(63) Continuation-in-part of application No. 15/776,255, filed on May 15, 2018.
(60) Provisional application No. 62/211,203, filed on Aug. 28, 2015.

(51) Int. Cl. A61K 35/19; A61K 45/06; A61K 47/26; A61P 7/04

(52) U.S. Cl. CPC A61K 35/19; A61K 45/06; A61K 47/26; A61P 7/04

(57) ABSTRACT

Compositions and methods for providing one or more platelet-derived hemostatic agents to a treatment site are provided. Compositions and methods may be directed to bandages, carrier materials, and closure devices. Compositions and methods may be directed to treatment of neoplasias, including cancers. The platelet-derived hemostatic agents may include chemotherapeutic agents.

### CROSS-REFERENCE TO RELATED APPLICATIONS

This application is a Continuation-In-Part of U.S. Pat. Application No. 15/776,255 filed on May 15, 2018, which is the National Stage application of International Application No. PCT/US2016/048846, filed on Aug. 26, 2016. International Application No. PCT/US2016/048846 claims priority to U.S. Provisional Pat. Application No. 62/211,203, filed on Aug. 28, 2015. The content of each of the applications listed above is incorporated herein by reference in its entirety.

### FIELD OF THE INVENTION

The present invention relates to the field of medicine. More specifically, the invention relates to hemostatic products, methods for controlling bleeding and improving healing, and hemostatic products containing bioactive agents, such as chemotherapeutic agents for treatment of subjects in need.

### BACKGROUND OF THE INVENTION

### Description of Related Art

Bleeding is a significant issue related to patient care. Bleeding as a result of trauma, including all types of trauma from scratches of the skin to blunt force trauma to trauma as a result of surgery, has traditionally been treated with bandages, either alone or in conjunction with compression, antibiotics, or hydrogels, with sutures or staples following surgery, and with liquid cyanoacrylate-based adhesives. However, there are disadvantages to these materials and modes of intervention. For example, the materials used typically do not trigger the body’s natural responses, such as hemostasis, to stop bleeding. The materials also might need to be frequently changed by medical practitioners, the subject who was treated, or others who are caring for the subject. In addition, in situations where surgical intervention is required, loss of blood before surgery is possible and could reduce the subject’s ability to mount a sufficient natural response to stop or slow bleeding.

In addition to better materials and modes of treatment, improvements in healing times are also needed in the art. Reducing healing time for traumas that involve bleeding permits the subject’s metabolism, endocrine system, and immune system, among other bodily systems, to return to normal function more quickly, thus improving the subject’s overall return to normal. Reduced healing time also allows the subject to return to his or her regular daily routine more quickly. In addition to the benefits for patients from faster recover times, it is also beneficial for the subjects to heal faster so that the subjects can be discharged from a medical facility at an earlier time, with a lower rate of readmission.

Platelets are important components of blood that are crucial for blood clotting and wound healing. Platelets are formed in the bone marrow as fragments of megakaryocytes. They are discoid-shaped, colorless bodies that are present in blood at a concentration of 150,000-50,000 per microliter (µl). Platelets play a crucial role in hemostasis, and they are the first line of defense against blood escaping from injured blood vessels. When bleeding from a blood vessel that has been ruptured occurs, platelets gather at the rupture site and initiate clot formation to block leakage of blood from the site. The process of clot formation is initiated when platelets circulating in the bloodstream contact collagen, which is exposed on the surface of subendothelial cells, following rupture of the endothelial lining of the vessel. Circulating von Willebrand factor (VWF) and VWF exposed on the surface of the subendothelial cells, binds to the surface of the platelets, enhancing the strength of binding to the subendothelial cells and causing the bound platelets to bind to circulating platelets. As the process continues, a platelet plug is formed at the site of rupture, blocking leakage of blood from the injured vessel. Concurrently, the adhered platelets release clotting factors and other factors that result in formation of a fibrin mesh that strengthens the plug until the vessel is repaired.

Platelets contain a number of important growth factors within their alpha granules that contribute to the process of hemostasis and wound healing. Studies have found that growth factors, such as platelet derived wound healing factors (PDWHF), platelet-derived growth factor (PDGF), transforming growth factor (TGF), and insulin growth factor (IGF), among others, are important in different stages of the wound-healing cascade and greatly influence mitogenic and cellular differentiation activities as well as helping cells to localize to the area of the wound.

### BRIEF DESCRIPTION OF THE DRAWINGS

FIG. 1 is a bar plot of the AUC from aggregation experiments for platelets (at a concentration of 250,000 platelets per µL) treated with collagen (10 µg/mL) and various concentrations of eptifibatide (“Epti”).

FIG. 2 shows the effect of eptifibatide at various concentrations on whole blood using T-TAS® technology.

FIG. 3 shows the effect of thrombosome (“Tsomes”) supplementation (approximately 200,000/µL) on whole blood with and without various concentrations of eptifibatide using T-TAS® technology.

FIG. 4 is a bar plot of the occlusion time for the data sets from FIG. 3.

FIG. 5 is a bar plot of the AUC for the data sets from FIG. 3.

FIG. 6 shows that thrombosomes (various lots) occlude in the presence of eptifibatide in platelet-poor plasma (PPP).

FIG. 7 is a bar plot of the AUC for data sets from FIG. 6. Replicate data sets from FIG. 6 are shown as averages.

FIG. 8 is a bar plot of the occlusion time for the data sets from FIG. 6. Replicate data sets from FIG. 6 are shown as averages.

FIG. 9A shows the effect of tirofiban alone, or with random donor platelets (RDP) or thrombosomes on platelet occlusion using T-TAS® technology.

FIG. 9B is a bar plot of the occlusion time for data sets from FIG. 9A.

FIG. 10A shows the effect of eptifibatide alone, or with RDP or thrombosomes on platelet occlusion using T-TAS® technology.

FIG. 10B is a bar plot of the occlusion time for data sets from FIG. 10A.

FIG. 11 shows the effect of dosing thrombosomes on the bleeding time of mice treated with a supra pharmacologic dose of clopidogrel.

FIG. 12 shows the recovery of thrombus formation promoted by thrombosomes in whole blood in the presence of ASA (200 micromolar), cangrelor (1 micromolar), AP2 6F1 (40 micrograms), as measured by occlusion time on the T-TAS AR chip coated with thromboplastin and collagen.

FIG. 13 shows the recovery of thrombus formation promoted by thrombosomes in whole blood in the presence of ASA (200 micromolar), cangrelor (1 micromolar) and 6F1 (40 micrograms/mL), as measured by occlusion (pressure) over time.

FIG. 14 shows the effect of thrombosomes supplementation to aspirin-(ASA-)inhibited whole blood (500 micromolar) on the interaction with plastic immobilized porcine collagen under high shear, as measured by AUC.

FIG. 15 shows the effect of thrombosomes supplementation to aspirin-(ASA-)inhibited whole blood (500 micromolar) on the interaction with plastic immobilized porcine collage under high shear, as measured by occlusion (pressure) over time.

FIG. 16 shows the effect of thrombosomes supplementation to aspirin-(ASA-)inhibited whole blood (100 micromolar) on the interaction with plastic immobilized porcine collage under high shear, as measured by AUC.

FIG. 17 shows the effect of thrombosomes supplementation to aspirin-(ASA-)inhibited whole blood (100 micromolar) on the interaction with plastic immobilized porcine collage under high shear, as measured by occlusion (pressure) over time.

FIG. 18 shows the effect on peak thrombin of thrombosome supplementation to normal and aspirin-inhibited plasma.

### DETAILED DESCRIPTION

Reference will now be made in detail to various exemplary embodiments of the invention. It is to be understood that the following discussion of exemplary embodiments is not intended as a limitation on the invention, as broadly disclosed herein. Rather, the following discussion is provided to give the reader a more detailed understanding of certain aspects and features of the invention.

Before embodiments of the present invention are described in detail, it is to be understood that the terminology used herein is for the purpose of describing particular embodiments only, and is not intended to be limiting. Unless defined otherwise, all technical and scientific terms used herein have the same meaning as commonly understood by one of ordinary skill in the art to which the term belongs. Although any methods and materials similar or equivalent to those described herein can be used in the practice of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “the platelet-derived hemostatic agent” includes a plurality of such platelet- derived hemostatic agents and reference to “a saccharide” includes reference to one or more saccharides, and equivalents thereof known to those skilled in the art. Furthermore, the use of terms that can be described using equivalent terms include the use of those equivalent terms. Thus, for example, the use of the term “subject” is to be understood to include the terms “patient”, “person”, “animal”, “human”, and other terms used in the art to indicate one who is subject to a medical treatment. Likewise, use of the term “tumor” is to be understood to encompass all types of dysregulated cell growth, and to be equivalent to the terms “cancer” and “neoplasia”. The use of multiple terms to encompass a single concept is not to be construed as limiting the concept to only those terms used.

Products and methods are described herein for controlling bleeding, improving healing (including healing of patients with solid tumors). Exemplary products and methods include platelet-derived hemostatic agents. The products and methods described herein are directed toward embodiments that aid in the closure and healing of wounds. These embodiments can further include delivery of anti-tumor agents to injured solid tumors. This disclosure reports the discovery that some of these products, such as bandages containing a platelet-derived hemostatic agent, lead to unexpectedly superior rapid healing with reduced scarring. In certain embodiments, the tools and procedures may be used in conjunction with other devices.

The embodiments disclosed herein may include lyophilized platelet-derived hemostatic agents. As disclosed herein, the terms “lyophilized platelet product”, “platelet- derived hemostatic agent”, “platelet-derived material”, “platelet-derived particles”, “platelet- derived product”, and “hemostatic agent” are used interchangeably. These terms are not to be interpreted as including naturally-occurring platelets or platelet microparticles. Instead, the terms are to be understood as limited to platelets that have been treated in some manner to change one or more physical characteristic of the platelets (e.g., as a result of lyophilization). In certain embodiments, the platelet-derived particles may comprise platelet-derived products that have not been activated.

In certain embodiments, a platelet-derived hemostatic agent may be delivered to a wound on the surface of or in the interior of a patient. In various embodiments, the platelet- derived hemostatic agent can be applied in selected forms including, but not limited to, adhesive bandages, compression bandages, liquid solutions, aerosols, matrix compositions, and coated sutures or other medical closures. In embodiments, the hemostatic agent may be administered to all or only a portion of an affected area on the surface of a patient. In other embodiments, the hemostatic agent may be administered systemically, for example via the blood stream. In embodiments, an application of platelet-derived hemostatic agent can produce hemostatic effects for 2 or 3 days, preferably 5 to 10 days, or most preferably for up to 14 days.

### Adhesive Bandages

Certain embodiments may include adhesive-type bandages with one or more portions of the adhesive bandage incorporating one or more platelet-derived hemostatic agents, or being impregnated with one or more platelet-derived hemostatic agents. In certain embodiments, the one or more platelet-derived hemostatic agents may be in direct contact with a treatment site.

Certain embodiments that include adhesive bandages may promote control of bleeding and healing of a wound. The adhesive bandage may include a treatment region, such as a gauze pad, which includes a platelet-derived hemostatic agent. The platelet-derived hemostatic agent may be in various forms including, but not limited to, particulate, powder, solution, gel, and matrix. The platelet-derived hemostatic agent may be retained within the treatment region by a type of container or receptacle. In certain embodiments, the container or receptacle provides direct access for the platelet-derived hemostatic agent to the treatment site. A mesh or other material with openings, such as, but not limited to interconnected strands, filaments, or strips of synthetic or natural material, may make up the container or receptacle and may allow contact for the platelet-derived hemostatic agent to contact the treatment site. These configurations allow for retention of the platelet-derived hemostatic agent within the treatment region. In alternative embodiments, the platelet-derived hemostatic agent may diffuse through a membrane, wall of the container, or receptacle, etc. to reach a treatment site. In certain embodiments, the treatment region may be permeable to liquid. This may allow blood to enter the treatment region, and come into contact with the platelet- derived hemostatic agent, thus initiating the clotting process.

In certain embodiments, a pad of an adhesive bandage may be coated with, impregnated with, or otherwise retain a platelet-derived hemostatic agent for application to a treatment site. In certain embodiments the entire adhesive bandage may be coated with, impregnated with, or otherwise retain a platelet-derived hemostatic agent for application to a treatment site. A rigid or semi-rigid support attached to the treatment region may provide for an application of pressure between the platelet-derived hemostatic agent and the treatment site.

Heating action associated with certain bandage forms may have the unexpected benefit of causing the platelet-derived particles to more effectively begin the reconstructive process. Heating may be caused by a chemical, electrical or other source. In certain embodiments, heat may be applied by an outside source of energy, such as infrared or other light source.

In certain embodiments, a bandage may be configured to deliver platelet-derived materials, as well as one or more biologically beneficial agents such as drugs (e.g., anti-tumor/chemotherapeutic agents), minerals, amino acids, pH modifiers, anti-microbials (e.g., antibacterials and antifungals), growth factors, and enzymes to the subject being treated. In certain embodiments, the platelet-derived materials, as well as one or more biologically beneficial agents, may be delivered as microencapsulated agents incorporated in adhesive backing. In certain embodiments, microencapsulated agents may be available in a gel matrix in a treatment region, accessible to the wound through pores or perforations, or using conventional transdermal technologies.

A bandage system may be a sealed system. Such systems are shown in U.S. Pat. No. 8,900,209, which is hereby incorporated by reference in its entirety. In certain embodiments, a bandage may include a seal, an external barrier (or top layer), a reservoir, an adhesive backing, and a permeable film (or bottom layer). The reservoir may be formed between the upper and lower layers (e.g., being hermetically sealed around the perimeter), but may also be a separate element of the bandage system, or contained within a continuous layer.

The external barrier (top layer) may be selected to be non-permeable to gases, liquids, and/or other materials. The permeable film (bottom layer) may be permeable to gases, liquids and/or other materials. The reservoir may store one or more platelet-derived materials when the bandage system is worn by a user. The platelet-derived material may be controllably released to the user through the permeable film.

In certain embodiments, the bandage system may also provide therapeutic and/or beneficial gases to a user. The amount of gas released to the user while wearing the bandage system may vary according to the concentration of the gas contained within the reservoir and the material used as the permeable film. Other factors such as temperature and atmospheric pressure may also affect the amount of gas released to the user.

The amounts of platelet-derived material delivered to the user may be influenced by the diffusion rates of the relevant material through the permeable portion, concentration range, and the length of time the bandage system is used.

Certain embodiments may also provide for vacuum treatment of wounds with bandages and platelet-derived materials. See U.S. Pat. Application Publication No. 2014/0330226, which is hereby incorporated by reference in its entirety. A bandage may be provided for use with a debrided wound. A bandage may include a layer adjacent to a wound, or surface of a wound. The bandage may also include a cover for placement over a wound and a structure between the cover and the layer adjacent a wound surface to create a vacuum space. The vacuum space or any other area adjacent to the wound may include platelet-derived materials. A vacuum may be created in the vacuum space by a vacuum source in communication with the vacuum space. The bandage, therefore, may combine the healing properties of platelet-derived materials with the acceleration provided by the vacuum therapy.

To treat a wound, the treatment region of the adhesive bandage may be placed in contact with at least a portion of the treatment site. Pressure may be applied to adhere the adhesive to the patient. The pressure may or may not be sufficient to restrict the bleeding itself, and the pressure can be combined with heat to speed up certain processes. After treatment, the adhesive bandage may be removed. In certain embodiments, the treatment region may be encased in a mesh or other similar material, the treatment region may be removed cleanly from the wound, requiring little to no irrigation of the wound to remove unwanted materials from the treatment site.

The rate of bleeding control and healing may be affected by the amount and placement of the platelet-derived hemostatic agent. The platelet-derived hemostatic agent may be separated from a treatment site by a diffusion limiting membrane or other barrier. In this case, a slow, delayed, controlled, or other form of release of the platelet-derived hemostatic agent may be achieved.

### Compression Bandages

Certain embodiments may include a combination of compression bandage with a platelet-derived hemostatic agent. Certain embodiments may include compression-type bandages with one or more portions of the compression bandage incorporating one or more platelet-derived hemostatic agents, or being impregnated with one or more platelet-derived hemostatic agents. In certain embodiments, the one or more platelet-derived hemostatic agents may be in direct contact with a treatment site.

Certain embodiments that include compression bandages may promote control of bleeding and healing of a wound. A compression bandage may include a treatment region including a platelet-derived hemostatic agent. The platelet-derived hemostatic agent may be in various forms including, but not limited to, particulate, powder, solution, gel, and matrix. The platelet-derived hemostatic agent may be retained within the treatment region by a type of container or receptacle. In certain embodiments, the container or receptacle provides direct access for the platelet-derived hemostatic agent to the treatment site. A mesh or other material with openings, such as, but not limited to, interconnected strands, filaments, or strips of synthetic or natural material, may allow contact for the platelet-derived hemostatic agent to contact the treatment site. As with the bandages described above, some types of mesh that dissolve on contact with liquid may be employed to dissolve into the clotted material. In alternative embodiments, the platelet-derived hemostatic agent may diffuse through a membrane, wall of the container or receptacle, etc. to reach a treatment site. As with the bandages described above, in some embodiments, the platelet-derived material will be on one or more strands or layers of the compression bandage and other strands or layers may contain additional material to accelerate clotting, such as divalent cations.

As with the bandages described above, in certain embodiments, the treatment region of the compression bandage may be permeable to liquid. This feature may allow blood to enter the treatment region, and come into contact with the platelet-derived hemostatic agent, thus shortening the time for the clotting cascade to begin.

In certain embodiments, one or more portions of a compression bandage may be coated with, impregnated with, or otherwise retain a platelet-derived hemostatic agent for application to a treatment site. In certain embodiments the entire compression bandage may be coated with, impregnated with, or otherwise retain a platelet-derived hemostatic agent for application to a treatment site. A rigid or semi-rigid support attached to the treatment region may provide for an application of pressure between the platelet-derived hemostatic agent and the treatment site. To treat a wound, the treatment region of the compression bandage may be placed in contact with at least a portion of the treatment site. Pressure may be applied by application of the compression bandage to a patient. The compression bandage may be secured to itself or the patient to retain the compression bandage in place. After treatment, the compression bandage may be removed. In certain embodiments, the treatment region may be encased in a mesh or other similar material, the treatment region may be removed cleanly from the wound, requiring little to no irrigation of the wound to remove unwanted materials from the treatment site.

The rate of bleeding control and healing may be affected by the amount and placement of the platelet-derived hemostatic agent. The platelet-derived hemostatic agent may be separated from a treatment site by a diffusion limiting membrane or other barrier. In this case, a slow, delayed, controlled, or other form of release of the platelet-derived hemostatic agent may be achieved.

### Powder, Liquid, or Matrix Compositions

Platelet-derived hemostatic agents may be applied through various methods not requiring an adhesive or compression bandage. Examples include, but are not limited to, liquid bandages, aerosols, suspensions, putties, gels, particulates, and powders. The platelet- derived hemostatic agent may be applied directly to a treatment site. For example, the liquid, solution, matrix, powder, etc. may be directly applied to a wound. Freeze-dried platelets and platelet-derived hemostatic agents have many potentially useful components for treatment, such as, but not limited to, growth factors, clotting factors, and homing-in factors. The platelet-derived hemostatic agent may be used by direct application to, for example, broken bones, open wounds, and orthodontic sites.

A liquid bandage composition may be provided. Such a composition may include, in addition to a platelet-derived hemostatic agent, a coating composition that, when applied to the skin, forms a durable waterproof (i.e., impervious to water) flexible film. Typically, the liquid bandage composition is made immediately before use by mixing the platelet-derived hemostatic agent with a liquid carrier. The composition may include a resin or other matrix-forming material that forms a film upon application. The liquid bandage may be applied by coating skin with the composition and allowing the composition to form a film over the treatment area. Preferably, the film is durable and capable of remaining adhered to the skin for 1 to 3 days and more preferably up to about 5 days. The durability of the film may depend on the composition of the film, the properties of the skin, and the environmental conditions to which the skin and film are subjected. The properties of the film may depend on the choice and quantity of resin or matrix-forming material. The properties of the film may be adjusted as necessary by changing the ingredients of the composition as well as by varying the relative amounts of ingredients. Other ingredients (e.g., diluent, thickener, and adhesive) may be added to the composition, and these may also affect the properties of the film.

The composition may be, for example, in the form of a paste, cream, gel, liquid, or aerosol. The film may be opaque, translucent, or transparent. The composition may be used as a bandage or as artificial skin.

The composition may include a diluent. Any suitable type of diluent may be used. The composition may include more than one type of diluent. Any suitable quantity of diluent may be used. The composition may be in the form of an aerosol, packaged under pressure with a suitable gaseous propellant. The diluent may be evaporative. Preferably, the diluent is an alcohol, such as ethanol, which may readily evaporate. The alcohol may be denatured or non-denatured. Preferably, ethanol (denatured with IPA) may be present in the composition in an amount of about 20-80% weight by weight (w/w).

Any suitable type of resin or matrix-forming material may be used. The composition may include more than one type of resin or matrix-forming material. The resin or matrix-forming material may be of natural or synthetic origin. Any suitable quantity of resin or matrix-forming material may be used. The resin may be a natural alcohol-soluble resin. In certain embodiments, the resin or matrix-forming material may be present in an amount of about 5-30% w/w.

A plasticizer may also be present in the composition. The plasticizer may impart flexibility to the film and to hinder flaking of the film from the skin. The composition may include more than one type of plasticizer. Any suitable quantity of plasticizer may be used. A suitable plasticizer may be oil. The oil may be of animal, vegetable, mineral, or synthetic origin. The oil may be, for example, castor oil, pine oil, eucalyptus oil, ti-tree oil, rosehip oil, or soya bean oil, or a mixture thereof. Preferably, oil is present in the composition in an amount of about 1-70% w/w. Other plasticizers may include cellulosic preparations.

The composition may also include an adhesive for improving attachment of the film to the skin. Any suitable type of adhesive may be used. The composition may include more than one type of adhesive. Any suitable quantity of adhesive may be used. Preferably, the adhesive is a cellulosic preparation such as, for example, ethyl cellulose or sucrose acetate isobutyrate. Other additives may include anti-agglomeration agents, dispersing agents, thickeners, preservatives, fragrances, dyes, etc.

In certain embodiments, the film may be resistant to being washed off with water. The film may remain intact even when immersed in hot water. The film preferably bonds to the skin when immersed in saltwater. The film is preferably highly resistant to being rubbed off. The film may remain attached to the skin even if a shaver is scraped over the film. If more than one film layer is applied to the skin, the film layers may bond to one another. Preferably, the film enables the skin to breathe.

For aerosol embodiments, the platelet-derived hemostatic agent may be combined with a polymer that forms a water and/or air seal over the treatment area. The platelet- derived hemostatic agent may become embedded or dispersed within the polymer upon aerosolization and crosslinking of the polymer.

Compositions may also be useful for deep, granulating chronic wounds, such as decubitus ulcers, venous ulcers, or in the acute setting, gunshot wounds, chainsaw or power saw accidents, etc. The platelet-derived hemostatic agent may be suspended or dispersed within a polymer, such as dextran or other water-soluble polymers for application to a wound site.

In certain embodiments, suspension, putties, or other materials may be created using one or more platelet-derived hemostatic agents. In certain embodiments, plastic biomaterials may be created using one or more platelet-derived hemostatic agents. See U.S. Pat. No. 8,529,961, which is hereby incorporated by reference in its entirety. Plastic biomaterials may be created by adding plasticizer and/or cross-linking materials to powdered platelet-derived hemostatic agents. Degradation time can be controlled by varying the amount of cross-linking agent used in manufacture as well as the processing parameters. Plastic materials may range in physical properties from elastomeric sheets to plastic parts exhibiting the stiffness and tensile properties of bone. Growth factors may be present in the plastic biomaterials and may directly be involved in bone and soft tissue healing processes. PDGFs (platelet derived growth factors), TGFs (transforming growth factors), IGFs (insulin- like growth factors), and FGFs (fibroblast growth factors) give rise to the plastics’ osteogenic properties, encouraging the healing and formation of new bone. Likewise, these same growth factors are known to support the growth of new blood vessels.

A bone putty form of the platelet-based plastics comprising the platelet-derived hemostatic agents may fill bone defects. The biologically-active filler may degrade over 8-12 weeks, slowly bathing the injured bone with a concentration of natural growth and regenerative factors in physiological proportions. Other uses may include a scaffold for soft tissue repair, which degrades over 4-6 weeks, accelerating the healing of injured tendons and ligaments.

In certain embodiments, the platelet-derived hemostatic agent may be administered using a nebulizer, aerosolizer, or a vaporizer. The material can be applied to the patient directly, or sprayed onto bandages or equipment that might come in contact with the patient. In some embodiments, the platelet-derived material can be injected or sprayed inside of a patient’s nostrils. In an embodiment, the platelet-derived material may be administered through inhalation. In an embodiment, the platelet-derived material may be sprayed or otherwise applied onto a wound dressing such as gauze, sponge, or a bandage, or the like, which may be applied to a wound. In an embodiment, platelet-derived material may be applied to a wound dressing and the wound dressing may be a one or more of woven fabric, textile, or a plastic, such as PVC, polyethylene or polyurethane. A wound dressing may comprise a latex strip. A wound dressing may be skin-toned or transparent. In an embodiment, a wound dressing may comprise glass fibers along with one or more of organic fibers, nylon fibers, ceramic fibers, cotton fibers, and the like. In an embodiment, a wound dressing may be free of glass fibers and/or wooden fibers. A wound dressing may be free of chemically fixed platelets. In an embodiment, a wound dressing may contain chemically fixed platelet-derived particles. In an embodiment, a wound dressing may contain a mixture of chemically fixed and non-chemically fixed platelet-derived particles. In an embodiment, a wound dressing may include the platelet-derived material and be bioadsorbable and may not require any dressing changes. In an embodiment, spraying or wound dressing materials may include a concentrated platelet-derived material that may allow for faster healing. In an embodiment, wound dressings may be formulated into about 5 cm by 5 cm pads, or about 10 cm by 10 cm pads, or about 2.5 cm diameter, or about 5 cm diameter pads that may be placed on wounds or may be placed inside the mouth to heal oral wounds.

In certain embodiments, powders containing a platelet-derived hemostatic agent may be used as a clotting composition. A composition or system, as well as articles or methods may be provided for the enhancement of clotting in wounds with extravascular blood flow, especially where the surface of the tissue has been broken. The system may include a platelet-derived hemostatic agent alone and/or in combination with one or more biotolerable, porous particulates (with pores chosen of the appropriate size for the effect desired) applied to the surface of a wound with liquid blood thereon. The porous nature of the particulate material, either free-flowing or packaged or restrained on or in a surface, may enhance clotting. When combined with the platelet-derived hemostatic agent, these particles produce a synergistic, unexpectedly improved result beyond what would be expected for use of either component individually or the components in combination. Furthermore, the system may provide the benefits with minimal and/or reduced risk based on use of animal- based materials, i.e., they are not a significant source of infection themselves. Chemical or biochemical agents, such as additional clotting agents, therapeutic agents, antibiotics, clot strengthening agents (such as fibrous structural materials), and the like may optionally be included on, with, or within the porous particles. Where the porous particle clotting agents are used with animals, materials which are mildly repellant to the animal patient (without being toxic) may be included within the applied particle material to better ensure that the animal will not tamper with the wound during healing, a common problem with veterinary treatments. The particles may comprise such diverse materials as organics, metallics, inorganics, ceramics, and the like, both natural and artificial. It is generally preferred that the pore size distribution lies within a general range, and this range may vary from animal to animal and condition to condition, but generally falls within about 0.5-1000 µm, or about 1 to 1000 nm, or about 5 to 500 nm, depending upon the particular use.

A composition that may be used for the enhancement of the clotting of blood in animals, including mammals, avians, and reptiles, may include powder/particulate one or more platelet- or thrombocyte-derived hemostatic agents alone and/or in combination with one or more porous particulate materials, which may be applied to the wound when there is blood in a liquid or only partially clotted state (e.g., where it may wet the particles). The particles may be applied to the wound area either as a free flowing powder of the particles, a dry spray/aerosol of particles, a moist spray/aerosol of the particles, as an association of particles in or on a carrier (such as a web, tape, fabric, foam, reticulated foam, or film), and may optionally contain conventional clotting agents with the particles. The particle application may enable direct contact of the particles with the flow of blood, preferably without any non-clotting intermediate film or material between the blood at the site of the wound and the clotting particles. For example, the use of the particles on the surface of a film with that surface facing the wound may be acceptable. In that orientation, the blood would clot on the wound site. Alternatively, a fairly thick, but porous film may be used, and the blood may flow through the pores of the film (e.g., greater than about 0.1 mm thickness) to reach the porous clotting particles on a backside of the film. The clot may not occur on the wound site. Alternatively, an intermediate structure may be to have the particles located within a thin, light fibrous mass so that as the particles enhance clotting. The fibers may remain within the region of clotting and strengthen the clot. The fibers could also be used to assist in carrying optional materials (e.g., antibiotics) to the wound site. One type of desirable materials of this last format would have a woven, non-woven or knitted fibrous sheet (e.g., less than about 1 mm in thickness, e.g., about 0.05 to 0.5 mm, or about 0.1 to 0.5 mm thick) with the fabric having a porosity of at least about 30%, or about 30-95%, about 40-95%, or about 50-95% porosity, with at least a portion of the porosity filled with the clot enhancing particles described herein. The particles may be carried within the structure of the fabric or bonded to the fibers, filaments, or yams of the fibrous material (taking care not to completely fill the pores of the particles with any binder used).

The particles may generally have a size of from about 1 to 1000 micrometers, or about 1 to 500 micrometers, but the size may be varied by one ordinarily skilled in the art to suit a particular use or type of patient and depending on the ability of a carrier to support the particles with their optional selection of sizes. Examples of specific porous particulate materials useful in the practice of the present invention may include porous materials from within the classes of polysaccharides, cellulosics, polymers (natural and synthetic), inorganic oxides, ceramics, zeolites, glasses, metals, and composites. Preferred materials may be non-toxic and may be provided as a sterile supply. Polysaccharides may be preferred because of their ready availability and modest cost. The porous particulate polysaccharides may be provided as starch, cellulose and/or pectins, and even chitin may be used (animal sourced from shrimp, crab and lobster, for example). Glycosaccharides or glycoconjugates, which are described as associations of the saccharides with either proteins (forming glycoproteins, especially glycolectins) or with a lipid (glycolipid) are also useful. These glycoconjugates appear as oligomeric glycoproteins in cellular membranes. In any event, all of the useful materials must be porous enough to allow blood liquid and low molecular weight blood components to be adsorbed onto the surface and/or absorbed into the surface of the particles. Porosity through the entire particle is often more easily achieved rather than merely etching the surface or roughening the surface of the particles.

Ceramic materials may be provided from the sintering, sol-gel condensation, or dehydration of colloidal dispersions of inorganic oxides such as silica, titanium dioxide, zirconium oxide, zinc oxide, tin oxide, iron oxide, cesium oxide, aluminum oxide, and oxides of other metal, alkaline earth, transition, or semimetallic chemical elements, and mixtures thereof. By selection of the initial dispersion size or sol size of the inorganic oxide particles, the rate of dehydration, the temperature at which the dehydration occurs, the shear rate within the composition, and the duration of the dehydration, the porosity of the particles and their size can be readily controlled according the skill of the ordinary artisan.

With regard to cellulosic particles, the natural celluloses or synthetic celluloses (including cellulose acetate, cellulose butyrate, cellulose propionate, etc.) may be exploded or expanded according to techniques described in U.S. Pat. No. 5,817,381, incorporated herein by reference in its entirety, and other cellulose composition treating methods described therein, which can provide porous particles, fibers and microfibers of cellulose based materials. Where the porous materials, whether of cellulose or other compositions, have a size that may be too large for a particular application, the particles may be ground or milled to an appropriate size. This can be done by direct mortar and pestle milling, ball milling, crushing (as long as the forces do not compress out all of the porosity), fluidized bed deaggregation and size reduction, and any other available physical process. Where the size of the raw material should be larger than the particle size provided, the smaller particles may be aggregated or bound together under controlled shear conditions with a binder or adhesive until the average particle size is within the desired range.

Porosity may be added to many materials by known manufacturing techniques, such as 1) codispersion with a differentially soluble material, and subsequent dissolution of the more soluble material, 2) particle formation from an emulsion or dispersion, with the liquid component being evaporated or otherwise removed from the solid particle after formation, 3) sintering of particles so as to leave porosity between the sintered or fused particles, 4) binding particles with a slowly soluble binder and partially removing a controlled amount of the binder, 5) providing particles with a two component, two phase system where one component is more readily removed than another solid component (as by thermal degradation, solubilization, decomposition, chemical reaction such as, chemical oxidation, aerial oxidation, chemical decomposition, etc.), and other known process for generating porosity from different or specific types of compositions and materials. Where only surface porosity is needed in a particular clot promoting format, surface etching or abrasion may be sufficient to provide the desired surface porosity.

A particularly desirable and commercially available material may include polysaccharide beads, such as dextran beads which are available as SEPHADEX™ beads from Pharmacia Labs. These are normally used in surgery as an aid to debridement of surfaces to help in the removal of damaged tissue and scar tissue from closed wounds. The application of this type of porous bead (and the other types of porous beads to open wounds with blood thereon) has been found to promote hemostasis, speeding up the formation of clots, and reducing blood loss and the need for continuous cleaning of the wound area. Bleeding from arteries, veins and small capillaries, soft tissue, organs (e.g., liver, kidney, lungs and spleen) can be effectively managed, reduced and eliminated in most cases by application of the particles or beads according to the present invention.

The platelet-derived hemostatic agent and/or porous particles or porous beads may be directly applied to surfaces or held in place by pressure. The platelet-derived hemostatic agent and/or beads or particles may be free flowing or be supported on or in a containment system. For example, the platelet-derived hemostatic agent and/or particles may be adhered to the surface of a sheet or film which may be applied (e.g., contacted, wrapped, adhered, secured, affixed, or otherwise place into a position where blood on the wound area will be absorbed or adsorbed by the porous particles or porous beads) to areas of a wound with blood thereon. The platelet-derived hemostatic agent and/or porous particles may also be provided in a form where the porous particles or porous beads may be interspersed with fibers, filaments, or other particles in a self-supporting structure, entangled within the fibrous elements of a net, web, fabric, or sheet, embedded in a sheet or film (with the particles exposed to enable adsorption or absorption of blood in contact with the wound), a packet of material, with the particles or beads free-flowing within the confines of the packet. The terms particles and beads are not intended to denote any substantive difference in size, shape, or performance of materials and are not asserted as having any distinct differences within the practice of the present invention, but are merely alternative terms. The use of only one term does not intend that the other term is not equally applicable in the context in which the one term is used. The porous particles and porous beads may also be provided as part of a patch system, with a fibrous network associated with the particles to provide a high level of structural integrity and strength to the applied assembly over the wound, even before clotting has occurred. This would be particularly appropriate where the assembly was being used as a stitch replacement or true wound closure system rather than only promoting clotting.

The porous particles may easily be associated with or carry additional, but optional, clotting or wound treating materials or ingredients. For example, it would be desirable to provide the porous particles with antibiotics, antifungal agents (especially where application may be in a tropical environment), topical pain reducing medication, pharmaceuticals, antiinflammatory agents, tissue enzyme inhibitors (e.g., epsilon aminocaproic acid, to reduce tissue enzyme production that would weaken the blood clot), and the like. Existing materials that promote clotting or control bleeding would be particularly, such as fibrin, thrombin, fibrinogen, aprotinin, fibronectin, and factor XIII.

The preferred polysaccharide components for the porous particles and porous beads of the present invention may often be made from cross-linked polysaccharides, such as cross-linked dextran (poly[beta-1,6-anhydroglucose]). Dextran is a high molecular weight, water-soluble polysaccharide. It is not metabolized by humans, is non-toxic, and is well tolerated by tissue in most animals, including most humans. There have even been extensive use of solubilized dextrans as plasma substitutes. The SEPHADEX™ beads specifically mentioned in the description of particularly useful polysaccharides include dextran crosslinked with epichlorihydrin. These beads are available in a variety of bead sizes (e.g., 10 to 100 µm), with a range of pore size. It is believed that pore sizes on the order of from 5 to 75% of volume may be commercially available and can be expanded to from 5 to 85% by volume or manufactured with those properties from amongst the type of beads described above. The sizes of the pores may also be controlled to act as molecular sieves, the pore size being from 0.5% or 1 to 15% of the largest diameter of the particles or beads. The SEPHADEX™ beads may have controlled pore sizes for molecular weight cutoff of molecules during use as a sieve, e.g., with cutoff molecular being provided at different intervals between about 5,000 Daltons and 200,000 Daltons. For example, there are cutoff values specifically for molecular weight sizes of greater than 75,000 Daltons. This implies a particle size of specifically about 10 to 40 micrometers. These beads will rapidly absorb water, swelling to several times their original diameter and volume (e.g., from 1.2 to as much as five times their volume).

### Medical Closures

Closure devices may be used in combination with the platelet-derived hemostatic agents of the invention.

Closure devices may include, but are not limited to, sutures, staples, etc.

A platelet-derived hemostatic agent may be impregnated in or coated on the closure device, such as a filament of a suture. The suture may self-elute the platelet-derived hemostatic agent over time. Suture thread may be used with one or more needles. The filament of a suture may be coated or impregnated with the platelet-derived hemostatic agent. Various methods may be used to make sutures that carry a platelet-derived hemostatic agent. For example, such methods include direct extrusion as described in U.S. Pat. No. 6,596,296 to create filaments wherein the drug is uniformly distributed. Alternatively “core/sheath” and other multicomponent configurations may also be extruded as described in U.S. Pats. Nos. 7,033,603, 6,596,296, and 7,033,603, all of which are herein incorporated by reference. Alternate methods such as coating (e.g., spraying or dipping) all or part of the sutures or an “over the wire” extrusion as described in U.S. Pat. No. 6,858,222 may also be used. Additionally, gradients of the drug along the suture are sometimes preferred. These linear anisotropies are described in U.S. Pats. Nos. 6,596,296, 6,858,222, and 7,514,095, which are also hereby incorporated by reference. Additionally, sutures themselves can be made at least in part of materials that have desired activity in or around the site where the sutures are implanted or inserted. In certain embodiments, only selected portions of a suture may be coated or otherwise comprise the platelet-derived hemostatic agent. In certain further embodiments, portions of the sutures are selectively left unassociated with the platelet-derived hemostatic agent or are associated with a different active agent associated with a different portion of the suture. In other embodiments, temporally phased release of one or more drugs may be designed to coincide with known phases of wound healing as a means to reduce scaring and enhance the body’s natural wound healing processes. This may be accomplished, for example, by multilayer filaments as described in U.S. Pat. No. 7,033,603 or by using multiple means of incorporating the drug in the base material of the filament, such as simultaneous use of nanoparticles and microspheres within the same filament as described in U.S. Pat. No. 6,858,222. These references are incorporated herein by reference. In certain other embodiments, the suture surface may comprise one or more wells including the platelet-derived hemostatic agent. In other embodiments, all sections of sutures are coated with the platelet-derived hemostatic agent. The methods for applying platelet-derived hemostatic agents to sutures include, for example: (a) extrusion, (b) by directly affixing to the suture a formulation (e.g., by either spraying the suture with a polymer/platelet-derived hemostatic agent film, or by dipping the suture into a polymer/platelet-derived hemostatic agent solution), (c) by coating the suture with a substance such as a hydrogel which may in turn absorb the composition, (d) by interweaving formulation-coated thread (or the polymer itself formed into a thread) into the suture structure in the case of multi-filamentary sutures, and (e) constructing the suture itself with a drug-containing composition.

The structure of the suture may influence the choice and extent of application and/or incorporation of a platelet-derived hemostatic agent. The location of the incorporation of coating of the platelet-derived hemostatic agent may also influence/control the release kinetics of the platelet-derived hemostatic agent.

As sutures are made in a variety of configurations and sizes, the exact dose of platelet-derived hemostatic agent administered may vary with suture size, length, diameter, surface area, design and portions of the suture coated. Certain principles, however, can be applied in the application of this art. For example, in the context of coated sutures, dose can be calculated as a function of dose per unit area (of the portion of the suture being coated), or total drug dose. Total drug dose administered can be measured and appropriate surface concentrations of active drug can be determined. The total platelet-derived hemostatic agent administered may typically be substantially less than the equivalent systemic dose, because, by being associated with the suture, the platelet-derived hemostatic agent may be distributed directly in the vicinity of the target tissue rather than being evenly distributed through the whole body.

### Cancer Treatments

In certain embodiments, a platelet-derived hemostatic agent may be used to target and/or treat cancer. In these embodiments, the platelet-derived hemostatic agent comprises a chemotherapeutic agent (i.e., an anti-tumor/cancer agent). The platelet-derived hemostatic agent may selectively target tumor-associated vasculatures associated with cancer cells and/or tumors. Abnormalities in tumor blood vessels may define a pro-thrombotic environment capable of platelet activation that may be absent from the rest of the vascular network. Targeting and/or treatment of a tumor via systemic administration (e.g., infusion) may be possible based on the physiological predisposition of the platelet-derived hemostatic agent to adhere to areas of endothelial damage. In embodiments, a tumor could be irradiated or otherwise endothelially injured, causing bleeding of the tumor. The platelet-derived hemostatic agent that was or is being infused into the patient’s blood stream could then localize to the site of bleeding at the tumor and release the anti-tumor agent at that specific site.

The platelet-derived hemostatic agent may adhere to activated cancer cells in a dose-responsive manner. Systematic administration of the platelet-derived hemostatic agent may selectively target cancer damaged microvasculatures of various tissues. In accordance with the disclosure above, in embodiments, platelet-derived hemostatic agents may comprise (or be “loaded” with) therapeutics, cancer-treatment medicines, tumor-inhibiting viruses, or other substances capable of damaging, inhibiting the growth of, or killing neoplastic cells. Once a tumor or other neoplastic growth has been irradiated or physically injured, the loaded platelet-derived hemostatic agent can target the area of endothelial damage of the tumor or other neoplastic growth, thus treating the tumor or growth. This aspect of the invention is particularly well suited for treatment of solid cancerous tumors.

In other embodiments relating to treatment of cancers, a platelet-derived hemostatic agent can be delivered directly to the site of a tumor, or in embodiments a site where a tumor has been surgically removed. For example, currently, the use of laparoscopic surgery to treat gynecological tumors, cervical cancer, and gastrointestinal stromal tumors, to perform cystectomies to treat bladder cancer, and to perform colectomies to treat colorectal cancer is well known and widely practiced. While laparoscopic surgery is considered minimally invasive, bleeding at the site of removal of the tumor or the affected tissue or organ still occurs and must be treated for optimal outcome. In embodiments of the present invention, enhanced wound closure at the site of surgery can be accomplished by delivery of the platelet-derived hemostatic agent according to the present invention directly to the site via the laparoscope in any of the appropriate forms of the platelet-derived hemostatic agent discussed herein. Further, in situations where a tumor has been excised, but the surgeon has reason to believe that the entire tumor was not removed, a platelet-derived hemostatic agent loaded with an anti-tumor agent can be delivered to the tumor site. Delivery of such a platelet-derived hemostatic agent allows for delivery of anti-tumor agent directly to the remaining tumor cells and can improve the treatment of the tumor.

As such, the invention includes a method for treating neoplastic cells in a subject where the method comprises administering to the subject a platelet-derived hemostatic agent in an amount sufficient to kill or reduce the growth rate of at least some neoplastic cells in the subject, wherein the platelet-derived hemostatic agent comprises a chemotherapeutic agent that functions to kill or reduce the growth rate of the neoplastic cells. In embodiments, the neoplastic cells form a cancerous tumor. In embodiments, the method further comprises irradiating or physically injuring the neoplastic cells or the vasculature supporting them. The irradiating or physically injuring the cells or vasculature causes bleeding at the site of irradiation or injury. In embodiments, the platelet-derived hemostatic agent selectively targets tumor-associated vasculatures associated with neoplastic growths. The step of administering can be by way of systemic delivery of the platelet-derived hemostatic agent via the subject’s bloodstream, such as by injection or infusion. Alternatively, the step of administering is by way of direct application of the platelet-derived hemostatic agent to the neoplastic cells. In embodiments, the method can be considered a method of treating a subject having a neoplastic growth within his or her body.

Further, it is evident that the invention includes a platelet-derived hemostatic agent for use in treatment of a neoplasia. The platelet-derived hemostatic agent comprises platelet-derived material resulting from freeze-drying of a platelet composition that includes platelets containing a chemotherapeutic agent that functions to kill or reduce the growth rate of the neoplasia. In embodiments, the platelet-derived hemostatic agent is from a human while in other embodiments, the platelet-derived hemostatic agent is from a non-human animal. In view of the disclosure herein, it is evident that the invention includes the use of a platelet-derived hemostatic agent for use in treatment of a neoplasia, wherein the platelet- derived hemostatic agent comprises platelet-derived material resulting from freeze-drying of a platelet composition that includes platelets containing a chemotherapeutic agent that functions to kill or reduce the growth rate of the neoplasia. In embodiments, the use involves the use of a platelet-derived hemostatic agent that is from a human or from a non-human animal.

### Platelet-Derived Materials

In certain embodiments, the platelet-derived material may be in the form of a powder. The platelet-derived material may also be a liquid, a paste, a gel, or within a matrix. In one embodiment, a hydrogel mixture may be added to a wound dressing and may include the platelet-derived material. In an embodiment, a wound dressing may take the form of a collagen dressing and may include layered hydrogel with platelet-derived material mixture. In certain other embodiments, the platelet-derived material can be added to a hydrophilic mixture such as petroleum jelly or mineral oil. It can be applied in any manner used to coat materials. Of particular interest are embodiments where the platelet-derived material is added to or impregnated into a bandage. Such platelet-derived material may contain platelets or fragments of platelets, cross-linked platelets, or serum that is freeze-dried either in the bandage or applied to the bandage after freeze-drying.

In certain embodiments, the platelet-derived material may be sprayed, such as in an aerosol solution. In certain embodiments, the platelet-derived material may be pre-mixed with an application material.

In certain embodiments, the application material may be sterile water, blood or blood components (e.g., plasma), glycerol, saline, buffered saline, petroleum jelly, hydrogel, cellulose, hydroxy ethyl cellulose, hydroxy methyl cellulose, mineral oil, amyl acetate, benzalkonium chloride, castor oil, clove bud oil, ethyl alcohol, isobutane-propane (propellant), n-Butyl acetate, nitrocellulose, and combinations thereof.

In certain embodiments, the platelet-derived material may comprise one or more salts, such as phosphate salts, sodium salts, potassium salts, calcium salts, magnesium salts, and the like. The platelet-derived material may include material other than platelets, including but not limited to, sugars, such as monosaccharides and disaccharides (e.g., maltose, dextrose, mannose, trehalose, sucrose, polymers of sucrose, glucose); polysugars, such as Ficoll-70 and Ficoll-400; glycerol; triglycerides; polysaccharides; lipids; dextran; polyvinyl pyrolidine (PVP); starch; hydroxyethyl starch (HES); and the like. Yet other exemplary substances include biological molecules derived from human or animal sources, such as polypeptides (e.g., albumins such as bovine serum albumin and human serum albumin), casein, laminin, fibrinogen, and the like. The platelet-derived materials can be freshly obtained, but preferably they are freeze-dried to increase their longevity. The freeze- drying procedure may result in lysis of a certain number of platelets. In some embodiments, compositions of the invention may comprise, external to intact platelets, some or all of the components present in the interior of a platelet. In certain embodiments, the platelet-derived hemostatic agents may have a different size distribution than that found in normal blood. In these embodiments, these platelet-derived hemostatic agents may be sorted according to size, and certain portions of the size distribution may be employed for different uses. In certain embodiments, certain portions of the size distribution may exhibit better results depending on the use.

In an embodiment, the platelet-derived material can be derived or isolated from humans or non-human animals, such as, but not limited to, dogs, cats, horses, pigs, rabbits, monkeys, goats, rats, mice, bovines, sheep, and elephants or other wild animals. In all embodiments of the present invention, the source of or the subject to which the platelet- derived hemostatic agent can be a human or a non-human animal. One particular group of substances that may be present in a composition of the invention is chemical and biological compounds that function as drugs. Another group is substances that function as cellular nutrients. Other substances may be compounds that function as markers or reporter molecules, including fluorescent agents or contrast agents for diagnostic or medical procedures. Yet another embodiment includes substances that function as herbal supplements. In certain embodiments, the substances are anti-coagulants. Compositions included in embodiments may contain fibrin. Compositions according to embodiments that do not contain fibrin may provide an advantage over compositions known in the art, for example when the compositions of the invention are used to treat non-compressible wounds.

One aspect of embodiments of the freeze-dried platelet-derived hemostatic agents according to the invention, or rehydrated freeze-dried agents, and compositions comprising them, is that the process of making the platelet-derived hemostatic agent can create platelet microparticles, which can accelerate clot formation, likely at least in part by way of their ability to promote tenase and prothrombinase activities, thereby enhancing thrombin-generating capacity and promoting rapid clot development at the injury site. In addition, due to the fact that the compositions can comprise a platelet-derived material and can contain a number of important growth factors, they can also contribute to the process of wound healing and tissue regeneration. Studies have found that mitogenic lipids and growth factors, such as platelet derived wound healing factors (PDWHF), platelet-derived growth factor (PDGF), transforming growth factor (TGF), and insulin growth factors (IGF), among others, are important in different stages of wound-healing cascade and greatly influence mitogenic and cellular differentiation activities. Thus, in some embodiments, one or more of these factors are included in the platelet-derived hemostatic agents or are provided in the methods of treating.

One aspect of embodiments of compositions comprising platelet-derived hemostatic agents is a use when anticoagulants have been administered to a patient. Anticoagulants can include one or more of aspirin, warfarin, heparin, clopidogrel, ticlopidin, tirofiban, eptifibatide herbal supplements, and the like, as would be known by those of ordinary skill in the art. Without being bound by any theory, embodiments disclosed herein advantageously remain effective, even in the presence of anticoagulants. Certain embodiments may include dosing a patient who recently has been administered an anticoagulant with a platelet-derived hemostatic agent prior to surgery. Often, prior to undergoing surgery, a patient ceases administration of anticoagulants to avoid uncontrolled bleeding during the surgical procedure, which in some cases could lead to the patient’s death. Ceasing administration of anticoagulants may place the patient at risk of arterial or venous thromboembolism (e.g., ischemic stroke, myocardial infarction, pulmonary embolism, deep vein thrombosis, and the like) during the time the patient ceases administration of these anticoagulants and the surgery. A patient may be administered a platelet-derived hemostatic agent prior to surgery, preventing uncontrolled bleeding during the surgery. This aspect also prevents arterial or venous thromboembolism because the patient is not required to cease administration of anticoagulants prior to and during the surgical procedure. In certain embodiments, the platelet-derived hemostatic agent may be administered intravenously, subcutaneously, orally, buccally, transdermally, transmucosally, and the like. In certain embodiments, the platelet-derived hemostatic agent may be in the range of about 0.5 µm to about 5 µm in size, or more particularly about 0.9 µm to about 2.5 µm, and these platelet- derived particles may be delivered intravenously or infusibly.

In certain embodiments, the platelet-derived hemostatic agent may be derived using recombinant methods.

In some embodiments, the platelet-derived hemostatic agent may be in the form of a powder that may be isolated from blood and preserved as described in U.S. Pat. 7,811,558 and U.S. Pat. 8,486,617 and U.S. Pat. 8,097,403 and U.S. Pat. 4,994,367, which are incorporated herein by reference in their entirety. In embodiments, the platelet- derived hemostatic agent may lack platelet surface markers and may lack or be deficient in some characteristics. For example, they may lack surface markers that are necessary for anti- platelet drugs. In some embodiments, expired platelets may be used as the source or part of the source of the platelet-derived material.

Embodiments provide methods of making freeze-dried platelet-derived hemostatic agents. The method may comprise obtaining platelets, exposing the platelets to at least one saccharide under conditions that are sufficient for the saccharide to be taken into the platelets; adding a cryoprotectant to the platelets; and lyophilizing the platelets to produce a platelet-derived hemostatic agent. In certain cases, the platelets may be provided as a mixture from two or more sources, such as a mixture of two or more units of blood, preferably three to 5, more preferably 6 to 10 or more units obtained from random blood donors to a public blood bank. In other embodiments, such as embodiments where the platelets are intended to be used at a later date for infusion back into the donor, the platelets can be from a known source, and are thus considered autologous platelets for the purposes of the methods of treatment disclosed herein. More specifically, the platelets may be originally obtained from the ultimate recipient of the platelet-derived hemostatic agent. The platelets may be provided from a fresh source (i.e., in-dated platelets from blood obtained from a donor less than 6 days prior to freeze-drying), although out-dated platelets may be used in some situations, particularly for preparation of platelet-derived agents intended for use as a hemostat to aid in stopping bleeding at a particular site of injury, and for in vivo and in vitro diagnostics or research.

The platelets that are provided may be suspended in a salt buffer that comprises at least one saccharide, resulting in a composition containing particles comprising platelet surface markers. The salt buffer may be any buffer that maintains at least a majority of the particles comprising platelet surface markers in an intact, functional state while in the buffer. Preferably, the buffer maintains the composition at a pH of about 6.2 to about 7.8. Thus, the salt buffer may be an isotonic salt buffer comprising salts naturally encountered by platelets, such as those comprising sodium salts, potassium salts, calcium salts, and the like, and combinations of such salts.

Alternatively, in certain embodiments the platelet preparation may comprise one or more salts that platelets are not naturally in contact with. The identity of the salt(s) in the buffer are not critical so long as they are present in amounts that are not toxic to the platelets and maintain at least a majority of the platelets in an intact, functional state while in the buffer. Indeed, certain salt solutions can be used to alleviate the symptoms of shock and may be preferred when patient shock is a concern. Likewise, the buffering component may be any buffer that is non-toxic to the platelets and provides adequate buffering capacity to the composition at the temperatures at which the composition will be exposed during the method of the invention. Thus, the buffer may comprise any of the known biologically compatible buffers available commercially, such as HEPES, phosphate-buffered saline (PBS), and Tris- based buffers, such as TBS. Likewise, it may comprise one or more of the following buffers: propane-1,2,3-tricarboxylic (tricarballylic); benzenepentacarboxylic; maleic; 2,2- dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); 1,40piperazinebis-(ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic(beta-methyltricarballylic); 2-(N-morpholino)propane-sulfonic acid (MOPS); phosphoric; and N-tris(hydroxymethyl)methyl-2-amminoethane sulfonic acid (TES), as well as natural cellular buffers such as carnosine. Furthermore, the buffer system can provide buffering capacity at the range of pH 4 to pH 8.

The salt buffer may comprise at least one saccharide. The saccharide may be any suitable saccharide, including a monosaccharide or disaccharide or polysaccharide. The saccharide may be any saccharide that is compatible with maintenance of viability and function of platelets, and may be present in any amount that is not toxic to the platelets. In general, the saccharide can be any saccharide that is capable of passing through a cell membrane, such as the platelet membrane. Examples of suitable saccharides are sucrose, maltose, trehalose, glucose, mannose, and xylose. A preferred saccharide for use in the method of preparing platelet-derived particles may be trehalose. The saccharide may be present in the buffer in any suitable amount. For example, it may be present in an amount of about 1 mM to about 1 M. In some embodiments, it is present in an amount of from about 10 mM 10 to about 500 mM. In some embodiments, it may be present in an amount of from about 20 mM to about 200 mM. In certain embodiments, it may be present in an amount from about 40 mM to about 100 mM. In certain particular embodiments, the saccharide may be present in the buffer in an amount of at least or about any of the following concentrations: 40 mM, 50 mM, 60 mM, 70 mM, 80 mM, 90 mM, and 100 mM. Of course, in various embodiments, the saccharide may be present in different specific concentrations within the ranges recited above, and one of skill in the art can immediately understand the various concentrations without the need to specifically recite each herein. Where more than one saccharide is present in the buffer, each saccharide may be present in an amount according to the ranges and particular concentrations recited above.

The salt buffer may comprise other components, as long as those components do not interfere with the intended beneficial property desired from the platelets at the concentration in which they are present in the buffer. Thus, polymers, such as proteins and polysaccharides, may be included in the buffer. Likewise, alcohols, such as ethanol, or polyalcohols, such as glycerols and sugar alcohols, may be included. Similarly, organic solvents, such as dimethyl sulfoxide (DMSO), can be included. Further, coagulation or platelet inhibitors, such as heparin, EDTA, EGTA, citrate, and prostaglandin E (PGE), can be included.

In embodiments, the buffer may comprise a HEPES-Tyrodes buffer (e.g., 25 mM HEPES, 119 mM NaCl, 5 mM KCl, 120 mM NaHCO3) comprising about 50 - 150 mM trehalose, pH 6.8. In other embodiments, the buffer can further comprise 1 - 5 % (v/v) ethanol, pH 6.8.

The platelet-containing composition may be incubated, at least in part to permit loading of the saccharide or other components into the platelets. The composition may be incubated at a temperature above freezing for at least a sufficient time for the saccharide or other component to come into contact with the platelets. Thus, incubation can be at or about 1° C., 4° C., 10° C., 20° C., 22° C., 25° C., 37° C., 42° C., 50° C., 55° C., or greater. In embodiments, incubation may be conducted at 37° C. Furthermore, incubation can be performed for any suitable length of time, as long as the time, taken in conjunction with the temperature, is sufficient for the saccharide to come into contact with the platelets and, preferably, be incorporated, at least to some extent, into the platelets. In embodiments, incubation is carried out for at least or about 10 minutes, 20 minutes, 30 minutes, 40 minutes, 50 minutes, 60 minutes, 70 minutes, 80 minutes, 90 minutes, 100 minutes, 110 minutes, 120 minutes, 130 minutes, 140 minutes, 150 minutes, 160 minutes, 170 minutes, 180 minutes, or longer. In certain embodiments, incubation is performed at 20° C. to 42° C. for 100 minutes to 150 minutes. In embodiments where activated platelets are desired, incubation times approaching or exceeding 4 hours in the presence of trehalose may be used. To reduce the amount of activation and minimize loss of structural integrity; however, incubation times of less than 4 hours, such as 2 hours, may be more suitable.

In some embodiments, a method of making the platelet-derived hemostatic agents or compositions comprising them may comprise providing a material that contains platelets and/or microparticles, removing all or essentially all red and white blood cells that might be present in the material, adjusting the pH of the resulting platelet-enriched material to an acidic pH, separating platelets, microparticles, or both from all or essentially all other components present in the material, resuspending the platelets, microparticles, or both in a liquid, and lyophilizing the platelets and/or microparticles. In some embodiments, one or more agents that are typically included in lyophilization procedures, such as sugars, may be added to the platelets and/or microparticles before lyophilizing. Exemplary sugars include, but are not limited to, monosaccharides, disaccharides (e.g., sucrose, lactose, maltose, isomaltose, cellobiose, or trehalose), or polysaccharides. In particular embodiments, the method comprises sterilizing the lyophilized material using any known technique that is suitable for sterilizing lyophilized materials. For example, the method can comprise heating the lyophilized platelets and/or microparticles for a sufficient time and temperature to kill bacteria and viruses.For example, in an embodiment, the method can comprise making a composition comprising microparticles. The method can comprise: pre-activating platelets with platelet agonists such as TRAP, collagen, thrombin, or ionophores, then incubating the platelets for about 30 minutes at 37° C. Doing so activates the platelets prior to loading and lyophilization, which increases the relative percent of microparticles in the freeze-dried composition. A specific exemplary protocol for generating compositions with high relative proportions of microparticles (in this case, about 60-90% microparticles) comprises: collecting PRP into tubes; centrifuging at 1000 × g for 15 minutes; decanting the supernatant; suspending the pellet in 10 ml PBS containing 10 mM EDTA, pH 6.5, washing in PBSE, pH 6.5; resuspending the pellet in PMP buffer (137 mM NaCl, 4 mM KCl, 0.5 mM MgCl₂, 0.5 mM Na₂HPO₄, 5.5 mM glucose, 10 mM HEPES, 2 mM CaCl₂) to achieve a platelet concentration of 2.5 × 10⁹ platelets per ml; adding 15 µM SFLLRN and incubating at 37° C. for 10 minutes; centrifuging the mixture at 750 × g for 20 minutes; removing the supernatant and centrifuging it at 10,000 × g at 4° C. for 30 minutes; removing the supernatant and resuspending the PMP in the same volume of 150 mM trehalose buffer (9.5 mM HEPES, 0.05 M NaCl, 0.0048 M KCl, 0.012 M NaHCO₃, 0.15 M trehalose, 0.005 M glucose, pH 6.8); adding ¼ volume of 30% ficoll (w/v), aliquoting the liquid into 0.5 ml portions; and lyophilizing.

Various modifications of the basic procedure, based on the parameters disclosed herein, can be made to either increase the relative amount of platelet-derived hemostatic agent particles as compared to microparticles, or to increase the relative amount of microparticles as compared to platelet-derived hemostatic agent. It has been found that increasing the relative amount of platelet-derived hemostatic agent may improve the suitability of the compositions for in vivo infusion or injection treatment uses because the activation level of the composition is relatively low, and the composition shows a higher number of characteristics of normal, fresh or in-dated platelets.

The platelet-derived hemostatic agent can be re-constituted or re-hydrated (used interchangeably herein) by exposure to an aqueous liquid, such as water or an aqueous buffer. Alternatively, the platelet-derived hemostatic agent preparations can be used directly in methods of treating, diagnostic methods, or research methods as discussed herein. In certain methods the platelets can be chemically fixed or cross linked prior to lyophilizing the platelets. The platelet-derived hemostatic agent can also be rehydrated by mixture with serum, or added to freeze-dried serum and reconstituted. Various embodiments may also provide lyophilized serum as the one or more platelet-derived hemostatic agents. Cells may be spun out of solution to create lyophilized serum. The lyophilized serum may also have benefits and can be used in the products and methods described herein.

In certain embodiments, it may be difficult to dehydrate the platelets along with the serum since the platelets may lyse. Separately dehydrating the platelets and the serum may allow for combination of the separate powder products (platelets and serum), which may avoid lysing. The freeze dried “cakes” of powder may be combined together to provide the platelet-derived hemostatic agents used herein.

In an additional aspect, embodiments provide a method of treating a subject in need of platelets or one or more platelet functions. Embodiments of the method may comprise administering a platelet-derived hemostatic agent to a subject in need of platelets or one or more platelet functions. In embodiments methods of using platelet-derived hemostatic agents to treat injuries or wounds involving bleeding, where the platelet-derived hemostatic agents are capable of being administered to a patient in need by direct application (such as by topical administration) rather than as an infusion of fresh or in-dated platelets may be included. Embodiments of using the platelet-derived hemostatic agents to treat injuries or wounds involving bleeding, include situations where the platelet-derived hemostatic agent are administered to a patient in need by infusion or injection of the platelet-derived hemostatic agent rather than by infusion of fresh or in-dated platelets.

In an embodiment, the platelet-derived material may include disinfectants, antibiotics, therapeutics, and the like. In certain embodiments, the platelet-derived hemostatic agent may comprise super-fine iron particles or similar materials within the particles comprising platelet surface markers. In these embodiments, the platelet-derived hemostatic agent may be administered internally to a patient. Upon administering the platelet-derived hemostatic agent, the super-fine iron particles can then be located using imaging techniques that detect the presence of materials such as iron. In certain embodiments, the platelet-derived hemostatic agent may comprise radiopharmaceutical agents, which can be used to detect injury using imaging techniques. In certain embodiments, the platelet-derived hemostatic agent may comprise therapeutic agents. In these embodiments, the platelet-derived hemostatic agent comprising a therapeutic agent may be used to direct the therapeutic agent to certain areas of the body of a human or animal. In these embodiments, the targeted area of the body can be intentionally injured (or previously injured unintentionally). In these embodiments, the platelet-derived hemostatic agent comprising a therapeutic can vascularly find its way to the site of the injury, thus directing a healing agent or a therapeutic agent directly to the site of injury. One non- limiting example of this embodiment is delivery of an antibiotic to a site of injury in which a blood vessel is ruptured. Another example of this embodiment may include directing a therapeutic agent to a cancerous site. As discussed above, in this embodiment, a cancerous tumor may be purposefully injured, thus causing the body to naturally direct platelets to the tumor. In these embodiments, the platelet-derived hemostatic agent comprising a therapeutic or healing agent will also be directed to the injured site, thus directing the therapeutic or healing agent directly to the site of the tumor, thus destroying the tumorous cancer cells, as also discussed above.

In some embodiments, the platelet-derived hemostatic agent may be a crushed powder and the powder may be contained in a glass or plastic vial or ampoule, a bag, a semipermeable bag, housing, or any other kind of container. The powder may be formed by crushing or mechanically manipulating a lyophilized cake of platelet material and the crushing may take place after the cake has been placed in a container or before the cake has been placed in a container. In other embodiments, a cake of lyophilized platelet material may simply be rehydrated by addition of an aqueous liquid, such as sterile water. During lyophilization, the lyophilization apparatus may be equipped with a heating plate that may seal the end of a bag, semipermeable bag, wound dressings, or some other type of housing capable of being sealed with heat. In an embodiment, the lyophilization apparatus may be equipped with a gluing, epoxy, equivalent adhesive, or system that is otherwise capable of sealing a bag, a semipermeable bag, or other container. In some embodiments, lyophilization may seal the platelet-derived material while under vacuum and may also be in a sterile environment. In some embodiments, the platelet material is lyophilized in a glass or plastic vial, the vial stoppered, and the stopper affixed to the vial using a crimp-seal, as is well- known in the pharmaceutical arts. A sealed container, package, or bag of platelet-derived material may allow for a platelet-derived hemostatic agent to be manipulated into a powder when preparing the platelet-derived material.

Sealed platelet-derived materials may include a foil overwrap to protect from oxygen or moisture and may increase shelf stability. In some embodiments a lyophilizer is configured so the shelves move together to push stoppers or tops into individual vials or other containers while under vacuum. A lyophilizer may include heating bars that may be positioned on the bottom or upper portions of the shelves or walls such that a bag (or other container) may be pushed together and sealed under vacuum. A bag or other container to be sealed may be comprised of foil, PVC, or other material capable of self-adhering when heat is applied. A bag or other container sealed in a lyophilizer may also be sealed utilizing an adhesive material. Embodiments may include heat treatment or gamma radiation treatment before or after a platelet-derived material mixture is packaged. In some embodiments, the heat treatment of the platelet-derived material may result in a product that conforms to FDA standards.

In an embodiment utilizing a bag, or semipermeable bag, or other container, the container may be sized to be used for single dose applications or multiple (e.g., bulk) doses.

In an embodiment, the platelet-derived material may be used to treat burns, lacerations, or ocular, internal or oral injuries.

In an embodiment, the platelet-derived material is added to mineral oil or petroleum jelly, then a homogenizer, mixer, high-shear fluid processor, or an emulsifier may be utilized to create an emulsion. A microfluidizer may also be used to physically shear the platelet-derived material. In some embodiments, the pressure and flow rate of a spray apparatus may be utilized to shear the platelet-derived material during operation of a delivery device.

In an experimental application of aspects of embodiments contained herein, a cake of platelet-derived material was reduced into powder crystals through manipulation of the cake. The platelet-derived material was placed into an IWATA Eclipse HP-BCS Bottom Feed Airbrush with an air compressor for spraying powder and liquids, with a 0.6 mm orifice. The airbrush was used to dispense platelet-derived material in the form of powder onto a pre-wetted surface while varying the air pressure, which may allow for altering the configuration of the spray during application of the platelet-derived material.

In another experimental application of aspects of embodiments contained herein, a cake of platelet-derived material was rehydrated with water and the platelet-derived material was placed into an airbrush and a highly concentrated mixture of platelet-derived material was sprayed onto both wet and dry surfaces. After about 5-10 minutes, a dry glaze developed.

In another experimental application of aspects of embodiments contained herein, a cake of platelet-derived material was reduced into powder crystals. The platelet-derived material powder crystal material was dispensed into a small manual atomizer and nebulizer, which was puffed or sprayed onto a wet surface using an airbrush type device. The powder was absorbed into a wet layer and dried in about 1-3 minutes.

### EXAMPLES

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### Example 1. Prophetic Example

An animal model may be prepared for a bleeding experiment in a standard manner. Several test procedures will be followed for the experiment. A control group will provide a baseline for natural clotting without application of any outside compositions and/or methods. A first test group will receive application of a platelet-derived hemostatic agent as described herein. A second test group will receive application of one or more porous materials as described herein. A third test group will receive application of a platelet-derived hemostatic agent and one or more porous materials as described herein.

The test materials will be applied directly to a bleeding site on the animal model. The time and characteristics of the healing process will be determined.

As shown by the experiment, the use of a combination of the platelet-derived hemostatic agent and one or more porous materials can provide unexpected results over the use of both components alone. A synergistic effect is realized where the combination of components is statistically better than would be expected from use of the various components together.

The results of the following Examples demonstrate the impact of the thrombosomes product in an in vitro model of a patient taking antiplatelet drugs. Thrombosomes and other lyophilized platelet products are designed for infusion into a patient’s bloodstream following diagnosis of trauma or hemostatic failure. These drugs utilize multiple forms of platelet inhibition mechanisms which inhibit platelet response to adenosine diphosphate (ADP), arachidonic acid, fibrinogen and von Willebrand factor binding to name a few. These include drugs like aspirin, clopidogrel, ticlopidine, tirofiban ticagrelor, cangrelor and eptifibatide.

### Example 2. Protocols

Generation of Thrombosomes. Thrombosomes were prepared consistent with the procedures described in U.S. Pat. Nos. 8,486,617 (such as, e.g., Examples 1-5) and 8,097,403 (such as, e.g., Examples 1-3), incorporated herein by reference in their entirety.

### Transmission Light Aggregometry

Plasma samples with platelet or thrombosomes or combination of both are loaded into cuvettes and placed into the aggregometry chambers. The chambers warm the sample and provide constant stirring. The initiation of aggregation can be done by multiple types of inhibitor agents not limited to thrombin, ADP, collagen and any agent known to stimulate platelet aggregation. The samples can also have been taken as ex-vivo, or in-vitro supplemented with inhibitors. The instrument begins the assay by first recording the light transmission previous to stimulation for 2 minutes. The stimulant of interest is then introduced by the technician and the change in light transmission is recorded over time. The increase in light transmission corresponds to increase in platelet aggregation.

Evaluation by T-TAS® using an AR chip. AR chips are characterized by a single channel containing collagen and tissue factor; they can be used to analyze clotting and platelet function.

The T-TAS® instrument was prepared for use according to the manufacturer’s instructions. AR Chips (Diapharma Cat. # TC0101) and AR Chip Calcium Corn Trypsin Inhibitor (CaCTI; Diapharma Cat. # TR0101) were warmed to room temperature. 300 uL of rehydrated thrombosomes were transferred to a 1.7 mL microcentrifuge tube and centrifuged at 3900 g × 10 minutes to pellet. The thrombosomes pellet was resuspended in George King (GK) pooled normal human plasma or autologous plasma with or without autologous platelets to a concentration of approximately 100,000- 450,000/uL, as determined by AcT counts (Beckman Coulter AcT Diff 2 Cell Counter). 20 uL of CaCTI with 480 uL of thrombosomes sample in GK plasma were mixed with gentle pipetting. The sample was loaded and run on the T-TAS® according to the manufacturer’s instructions.

### Evaluation by T-TAS® Using a PL Chip

PL chips are run similarly to AR chips but this chip is only coated with collagen alone.

### Thrombin Generation

Reagent Preparation. For thrombin generation, the following materials were used from manufacturers, as follows: FluCa Kit (Diagnostica Stago, Cat. No. 86197), Thrombin calibrator (Diagnostica Stago, Cat. No. 86197), PRP Reagent (Diagnostica Stago, Cat. No. 86196), OCTOPLAS®, a solvent detergent treated human pooled plasma (Octapharma, Cat. No. 8-68209-952-04). All frozen reagents were thawed in a 37° C. water bath before use. All reagents were rehydrated with sterile water using the volume printed on the reagent labels. Approximately 2 min after rehydration, the reagents were mixed by inverting vials 5 times, so no chunks or powder left; vortexing was not used. This procedure was repeated approximately 10 minutes after rehydration. All reagents were incubated at room temperature for another approximately 10 minutes (total of approximately 20 min after rehydration). A 30% solution of OCTOPLAS® was prepared by mixing 4.66 ml of thrombosomes control buffer (Table B) with 2 ml of OCTOPLAS®.

TABLE B.

Thrombosomes Control Buffer

| | |
|---|---|
| Component | Concentration (mg/mL, except where otherwise indicated) |
| NaCl | 6.08 |
| KCl | 0.28 |
| HEPES | 2.47 |
| NaHCO₃ | 0.77 |
| Dextrose | 0.41 |
| Trehalose | 28.83 |
| Ethanol | 0.76% (v/v) |

Sample Analysis - Plate preparation and testing. For experiments containing thrombosomes, a thrombosomes dilution series was generated (dilutions of 194.4 K, 64.8 K, 21.6 K, and 7.2 K per µL were typically used; cell counts are determined by flow cytometry) for each the experimental thrombosomes and the reference thrombosomes. Thrombosomes were rehydrated unless indicated otherwise. The highest-concentration dilution (e.g., 194.4 k thrombosomes) was prepared by combining thrombosomes, OCTAPLAS®, and thrombosomes Control Buffer. The rest of the dilution series was prepared by serial 1:3 dilutions in OCTAPLAS®. For each test sample, 20 uL of PRP reagent was added to each sample well (of Immulon 2HB Clear, round-bottom 96-well plate (VWR, Cat. No. 62402-954)) and 20 uL of Thrombin Calibrator was added to each calibrator well. To each sample well and calibrator well, 80 uL of the each of the thrombosomes dilution series was added. Continue until the last dilution. The plate was then incubated in the Fluoroskan Ascent 96 well fluorescent plate reader (Thrombinoscope) (ThermoFisher Scientific) for 10 minutes. During this incubation phase, the FluCa solution was prepared by adding 40 µL of FluCa substrate to the 1.6 ml of thawed Fluo-Buffer, vortexing, and returning the solution to the water bath. When incubation was complete, the FluCa solution was added to the Fluroskan instrument according to the manufacturer’s instructions. The plate fluorescence was monitored for 75 minutes at an interval of 20 seconds and a temperature of 40-41° C.

### Example 3. GPIIb-IIIa Inhibitors

The results that follow demonstrate the impact of thrombosomes in an in vitro model of a patient taking a GPIIb-IIIa inhibitor. Eptifibatide, a common antiplatelet drug, competitively inhibits the GPIIb-IIIa receptor on platelets which interact with fibrinogen and von Willebrand factor.

Eptifibatide is a peptide therapeutic that blocks the fibrin binding role of GPIIb-IIIa receptor on platelets. The drug is typically administered via IV as a 180 µg/kg bolus followed by 2 µg/kg/min continuous infusion. The blood concentration of eptifibatide is typically about 1-2 µM. Bleeding time generally returns to normal within about 1 hour of drug stoppage.

Thrombosomes were prepared consistent with the procedure in Example 2. Transmission light aggregometry and T-TAS® experiments were carried out according to Example 2.

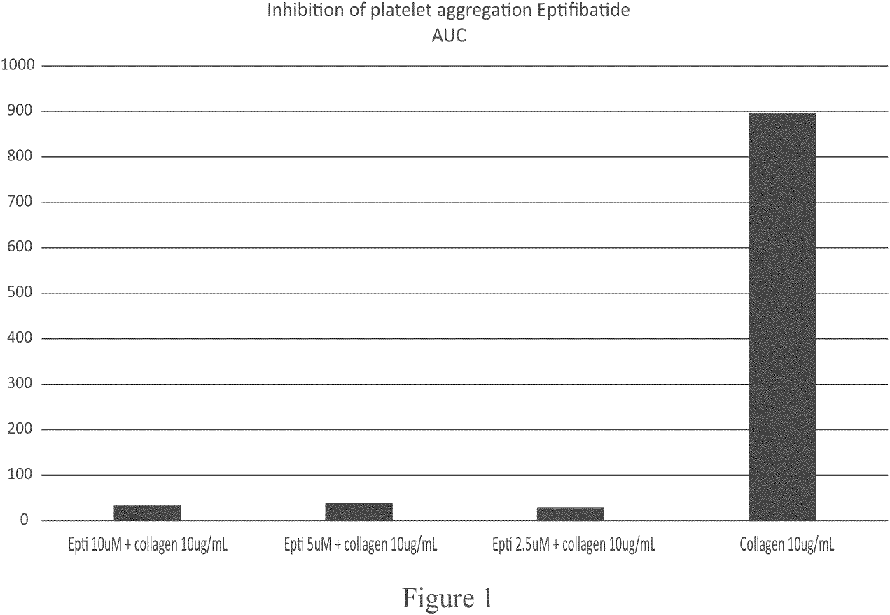

The aggregation of platelets (in platelet rich plasma) was evaluated using transmission light aggregometry. Eptifibatide completely inhibited collagen-induced (10 µg/mL) platelet aggregation in PRP at all concentrations tested, as detected by light transmission aggregometry in PRP. (FIG. 1).

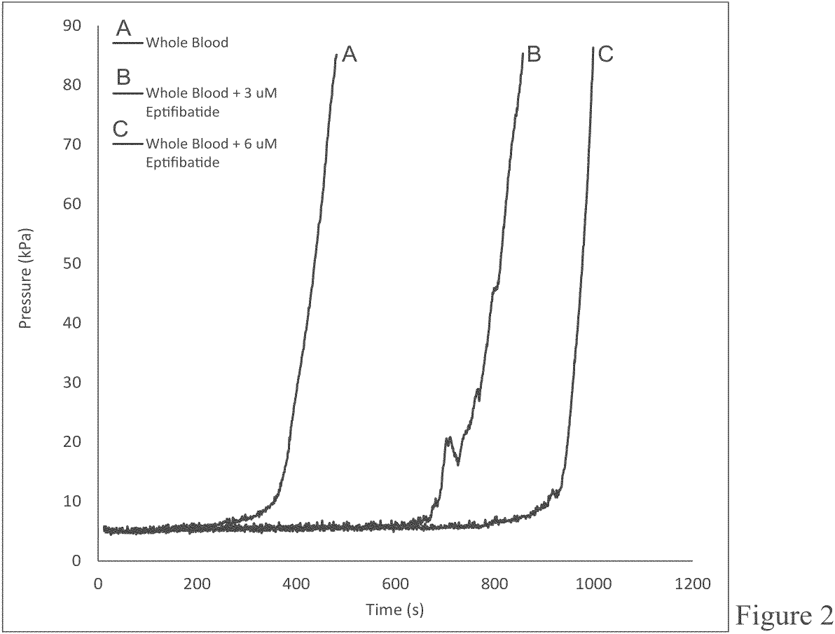

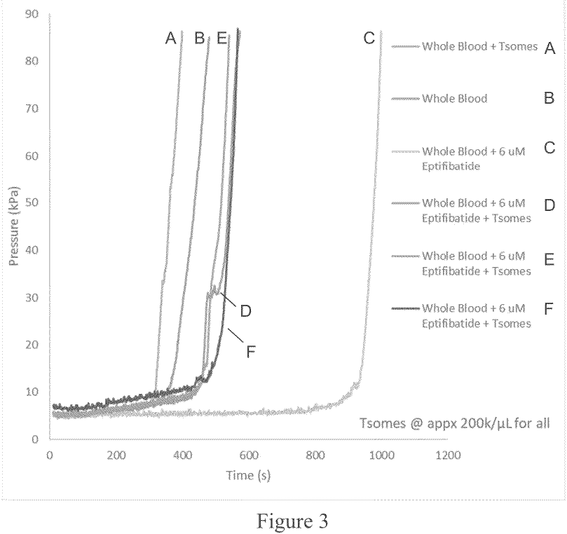

The effect of thrombosomes on shortening clotting times while in the presence of eptifibatide was also studied. The ability of thrombosomes to recover occlusion times was studied on the T-TAS® system. The T-TAS® system measures occlusion time under shear forces with collagen and thromboplastin stimulation. The whole blood profile of occlusion and AUC on the AR T-TAS® chip lengthened and decreased, respectively, with eptifibatide. Eptifibatide extended the occlusion time of whole blood on the T-TAS® AR Chip in a dose-dependent manner. In this experiment, whole blood occluded at 8 minutes, and the occlusion time was extended to 16 minutes with 6 µM eptifibatide (FIG. 2). Thrombosomes reversed the inhibitory effect of eptifibatide on thrombus formation. Eptifibatide inhibition of whole blood occlusion on the T-TAS® AR Chip was reversed by the addition of thrombosomes at approximately 200,000/µL (N=3). When thrombosomes (approximately 200k/µL) were added to the sample of whole blood inhibited with eptifibatide, the time to occlusion decreased to ‘normal’ at 9 minutes (FIG. 3).

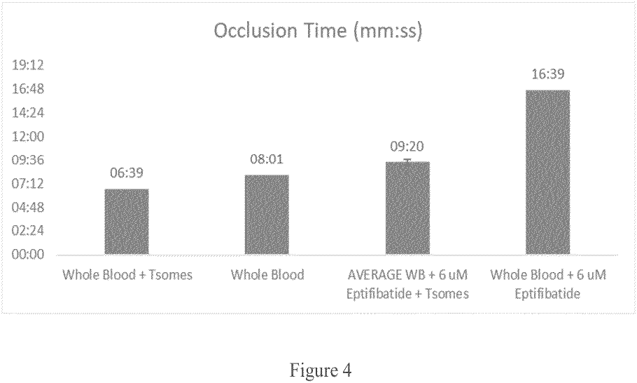

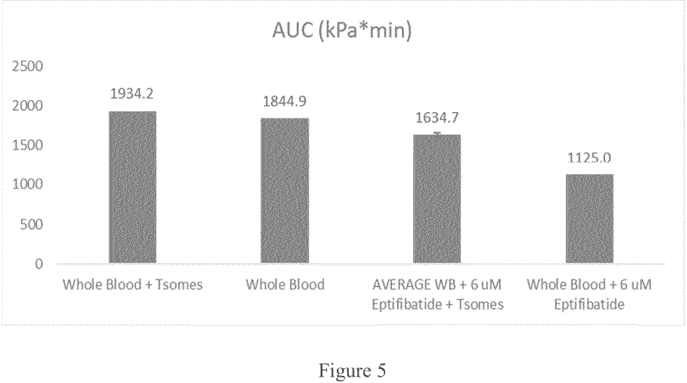

The area under the curve values with thrombosome treatment also increased with thrombosomes compared to that of normal whole blood samples. FIG. 4 demonstrates the time to of occlusion of the thrombosomes on AR T-TAS® chip with drug treatment; eptifibatide inhibition of T-TAS® AR Chip occlusion was nearly entirely reversed by the addition of thrombosomes (200,000/µL; N=3). In FIG. 5, the area under the curve values were indicative of thrombus formation, where thrombosomes returned inhibition by eptifibatide to normal levels; eptifibatide inhibition of platelet adhesion to and occlusion of the T-TAS® AR Chip is overcome by addition of thrombosomes (200,000/µL; N=3).

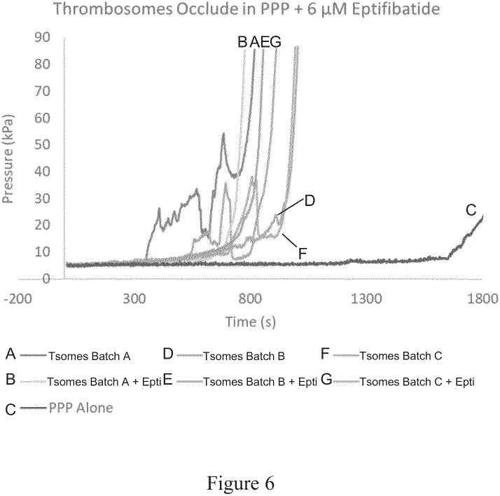

Thrombosomes, unlike platelets, are not inhibited in their ability to occlude under shear in the presence of eptifibatide (FIG. 6). FIG. 6 shows profiles of thrombus formation of various lots of thrombsomes on AR T-TAS® system were unchanged with eptifibatide treatment. Thrombosomes in platelet poor plasma (PPP) were flowed through the T-TAS® AR Chip with and without 6 uM eptifibatide. There was no effect of eptifibatide on thrombosome adhesion and occlusion. All thrombosome concentrations were approximately 300,000/µL.

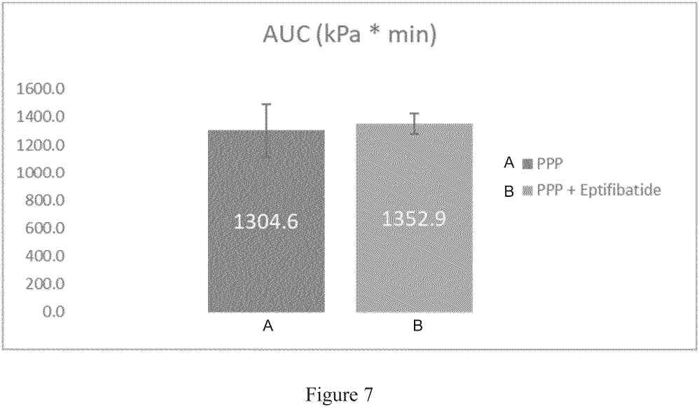

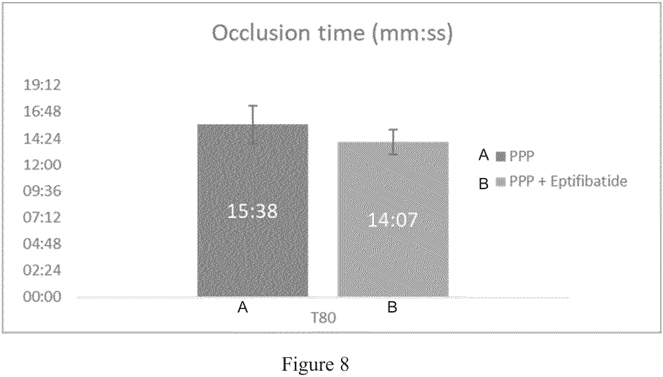

The AUC and occlusion values by T-TAS for thrombosomes (approximately 300,000/µL) in plasma was the same with and without eptifibatide (FIGS. 7- 8). FIG. 7 shows the area under the curve values were indicative of thrombus formation, and no changes were observed with eptifibatide in platelet-poor plasma. There was no effect of 6 uM eptifibatide on AUC of Thrombosomes T-TAS® AR Chip occlusion. FIG. 8 shows the time to occlusion of the thrombosomes on AR T-TAS® chip was unchanged with eptifibatide. There was no significant influence from 6 µM eptifibatide on thrombosomes occlusion time of the T-TAS® AR Chip in platelet-poor plasma.

Thrombosomes but not Random Donor Platelets (RDP) Reversed (Extended) Occlusion Times Induced by tirofiban in PRP.

Additional experiments were carried out with tirofiban. Thrombosomes were prepared consistent with the procedure in Example 2. T-TAS® was carried out according to Example 2. Random donor platelets were prepared from whole blood.

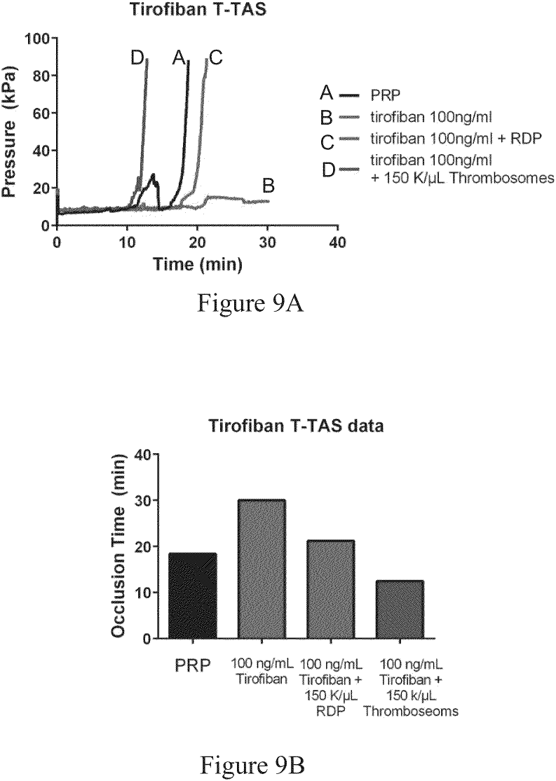

FIGS. 9A and 9B show that platelet rich plasma treated with 100 ng/mL tirofiban extended occlusion times from 18.43 to no occlusion on the T-TAS® flow system (collagen and tissue factor coated channel). The addition of 150k/µL of thrombosomes decreased the time back to 12.94 minutes but RDP only partially recovered at the same count.

Thrombosomes but not Random Donor Platelets Reversed (Extended) Occlusion Times Induced by Eptifibatide in PRP.

Additional experiments were carried out with eptifibatide. Thrombosomes were prepared consistent with the procedure in Example 2. T-TAS® was carried out according to Example 2. Random donor platelets were prepared from whole blood.

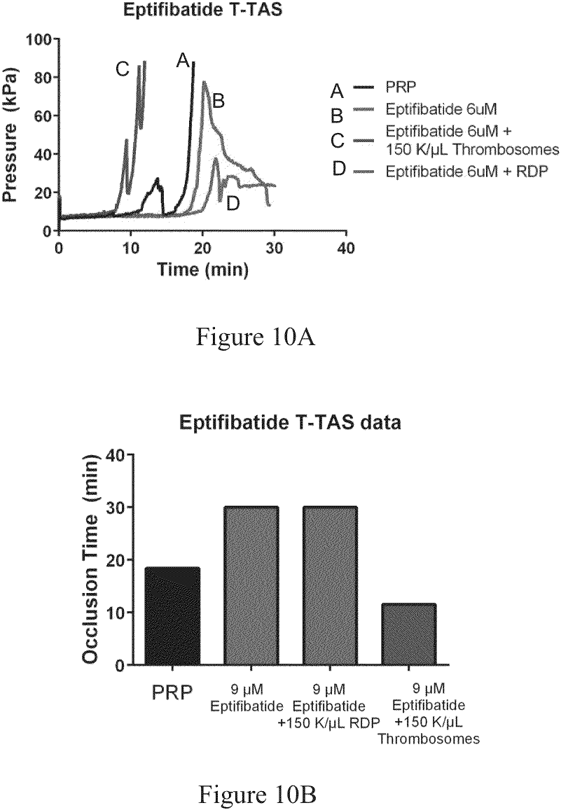

FIGS. 10A and 10B show that platelet rich plasma treated with 9 µM eptifibatide extended occlusion times from 18.43 to over 30 minutes on the T-TAS® flow system (collagen and tissue factor coated channel). The addition of 150k/µL of thrombosomes decreased the time back to 11.56 minutes but not occlusion seen with same number of RDP.

### Example 4. P2Y12 Inhibitors

Thrombosomes Restore Bleeding Time in NOD-SCID Mice Treated with Supra-pharmacologic Clopidogrel.

Additional experiments were carried out with clopidogrel. Thrombosomes were prepared consistent with the procedure in Example 2.

The mouse was treated with clopidogrel for 5 days. The mouse was anesthetized, the tail end was snipped off followed by thrombosomes being immediately administered. The time from tail snip to tail stop bleeding was recorded by visual inspection.

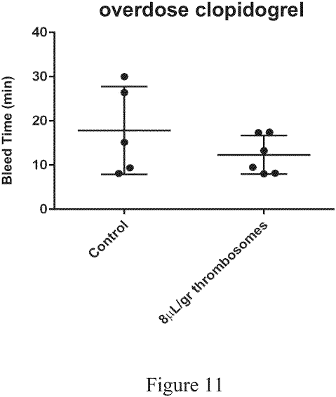

NOD/SCID mice were treated with ~ 3 times the clinical dose of clopidogrel for 5 days then assessed in the tail-snip bleed model. The bleed time (min) was extended to 17.8 minutes with clopidogrel treatment verses untreated at 9 minutes (data not shown). Treatment with 8 µL/gram of thrombosomes (1.8 × 10^9 particles/mL at 200 µL) decreased bleeding to 12.31 minutes (FIG. 11).

### Example 5. P2Y12 Inhibitor and Aspirin (ASA)

Additional experiments were carried out with cangrelor and aspirin. Thrombosomes were prepared consistent with the procedure in Example 2. Transmission light aggregometry, T-TAS®, and thrombin generation experiments were carried out according to Example 2. Cangrelor, like clopidogrel, blocks the P2Y12 (ADP) receptor on platelets. Cangrelor is used here as a representative of this class of drug.

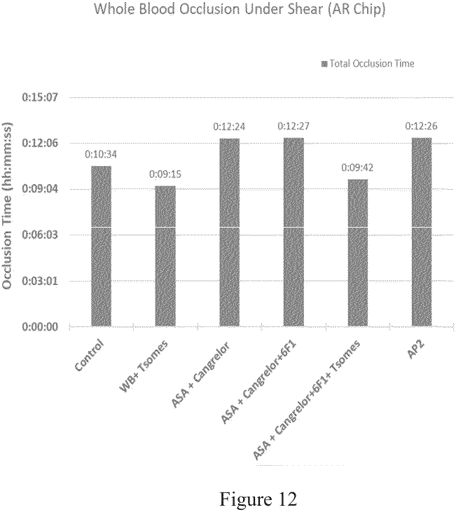

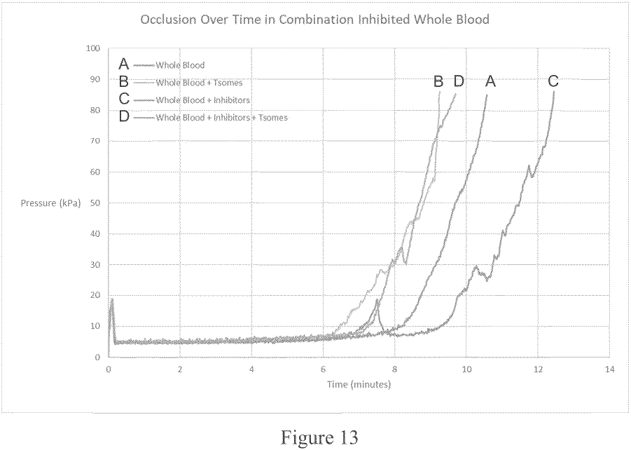

The effect of thrombosomes on the recovery of thrombus formation was evaluated using T-TAS® technology and an AR chip. FIG. 12 shows the occlusion time of whole blood treated with various combinations of thrombosomes (at a concentration of 250,000 thrombosomes per µL), aspirin (200 µM), cangrelor (1 µM), anti-Integrin alpha-2 (CD49B) antibody 6F1 (40 µg; see dshb.biology.uiowa.edu/integrin-alpha-2-alpha2beta1?sc=7&category=-107 for product/manufacturer information), and anti-GPIIb/IIIa receptor antibody AP2 (20 ug/mL; see kerafast.com/product/2010/anti-glycoprotein-gpiiiagpiib-complex-ap-2-antibody for product/manufacturer information). FIG. 13 shows the occlusion over time of untreated whole blood and whole blood treated with thrombosomes (at a concentration of 250,000 thrombosomes per µL), a mixture containing 6F1 (40 ug/mL; anti-CD49b), ASA (aspirin; 200 uM), and cangrelor (1 uM); or a combination thereof.

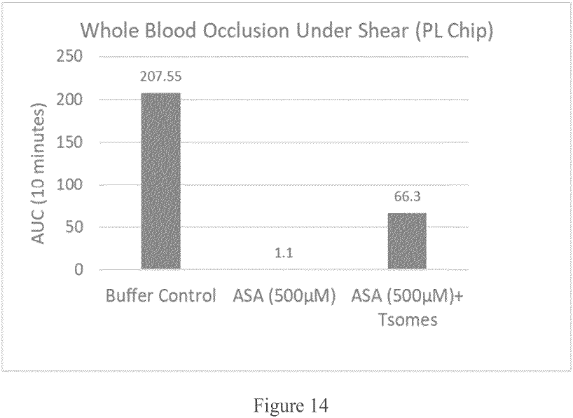

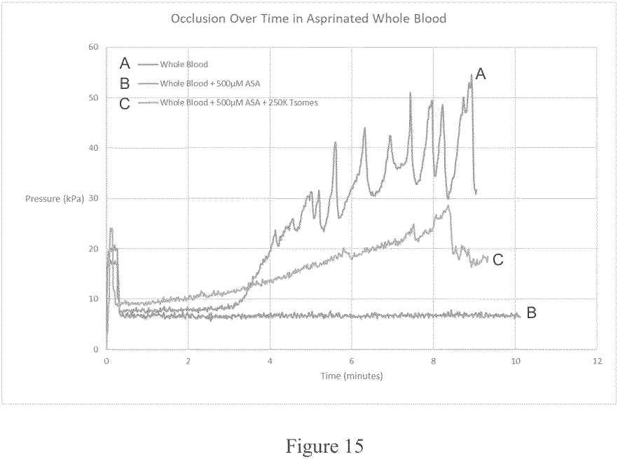

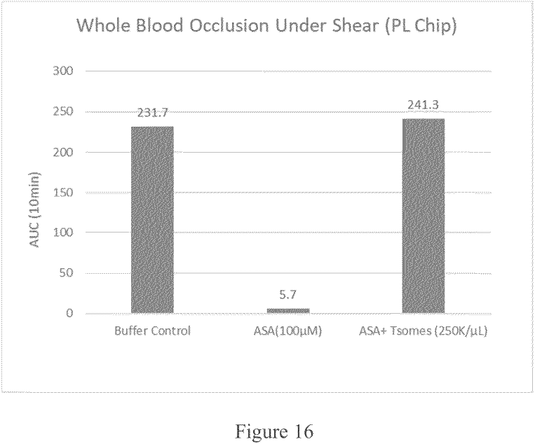

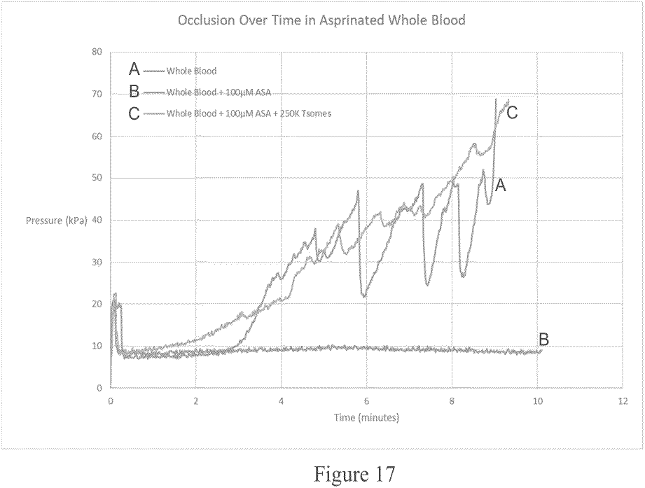

The effect of thrombosomes on the recovery of thrombus formation was also evaluated using T-TAS® technology and a PL chip. FIG. 14 shows the occlusion time of whole blood treated only with buffer, aspirin (500 µM), or aspirin (500 µM) and thrombosomes (at a concentration of 250,000 thrombosomes per µL). FIG. 15 shows the occlusion over time of whole blood, whole blood treated with aspirin (500 µM), or aspirin (500 µM) and thrombosomes (250,000/µL). FIGS. 16 and 17 show similar experimental data using 100 µM aspirin instead of 500 µM aspirin.

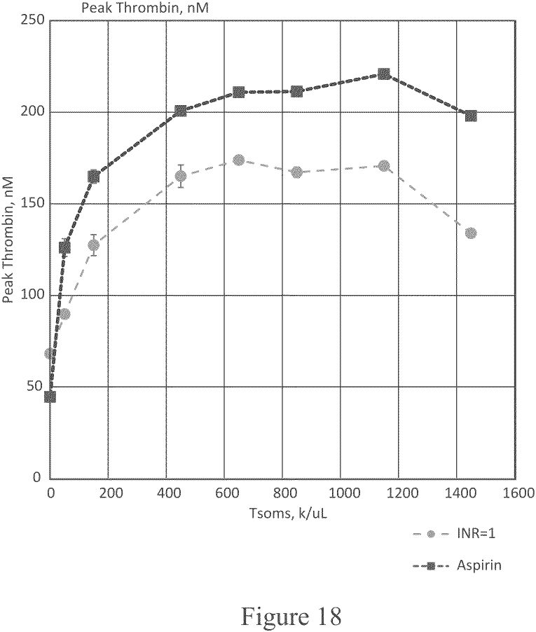

The effect of aspirin treatment (concentration) on thrombin generation was measured. Thrombosomes were evaluated at concentrations 1450, 1150, 850, 650, 450, 150, 50, and 0 k/uL in PPP from patients taking baby aspirin daily and standard plasma (INR = 1). FIG. 18 shows that the peak thrombin value of the aspirin plasma in absence of thrombosomes was below the normal range (about 45 nM; normal range is about 66-166 nM), but with thrombosomes addition, it came back to being within the normal range at even the lowest thrombosomes concentration used (50 k/µL). The values again were saturated at about 800 k thrombosomes and went up to 220 nM - 5 times the value of this plasma in absence of thrombosomes (increase from 45 to 220 nM).

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above. Furthermore, one having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

What is claimed is:

1. A method for controlling bleeding in a subject, said method comprising: administering to the subject a freeze-dried platelet-derived material comprising freeze-dried platelets, a buffer, a sugar, and one or more salts, wherein the subject has been administered an anti-platelet drug and aspirin, warfarin, or heparin such that the anti-platelet drug and the aspirin, warfarin or heparin are present in the subject at the time of the administering, wherein the freeze-dried platelet-derived material is effective as a hemostatic agent in the subject, and wherein the anti-platelet drug is one or more of clopidogrel, ticlopidine, tirofiban, and eptifibatide.

2. The method of claim 1, wherein the freeze-dried platelet-derived material is a rehydrated freeze-dried human platelet-derived material comprising rehydrated freeze-dried platelets in a liquid comprising the buffer, the sugar and the one or more salts, and wherein administering is by injection or infusion.

3. The method of claim 2, wherein the subject has been administered aspirin and the aspirin is present in the subject at the time of the administering.

4. The method of claim 3, wherein the subject has been administered clopidogrel, and the clopidogrel is present in the subject at the time of the administering.

5. The method of claim 3, wherein the subject has been administered tirofiban, and the tirofiban is present in the subject at the time of the administering.

6. The method of claim 3, wherein the subject has been administered eptifibatide, and the eptifibatide is present in the subject at the time of the administering.

7. The method of claim 3, wherein the subject has been administered ticlopidine, and the ticlopidine is present in the subject at the time of the administering.

8. The method of claim 1, wherein the subject has been administered clopidogrel, and the clopidogrel is present in the subject at the time of the administering.

9. The method of claim 1, wherein the subject has been administered tirofiban, and the tirofiban is present in the subject at the time of the administering.

10. The method of claim 1, wherein the subject has been administered eptifibatide, and the eptifibatide is present in the subject at the time of the administering.

11. The method of claim 1, wherein the subject has been administered ticlopidine, and the ticlopidine is present in the subject at the time of the administering.

12. The method of claim 3, wherein the sugar is selected from the group consisting of maltose, dextrose, mannose, trehalose, sucrose, a polymer of sucrose, glucose, a polysugar, a polysaccharide, starch, a hydroxyethyl starch, and combinations thereof.

13. The method of claim 12, wherein the sugar is trehalose at a concentration of 10 mM to 500 mM.

14. The method of claim 13, wherein the freeze-dried platelet-derived material further comprises a polysugar, wherein the polysugar is polysucrose, wherein the pH of the freeze-dried platelet-derived material is in the range of pH 6.2 to pH 7.8, and wherein the freeze-dried platelet-derived material is a rehydrated composition of freeze-dried platelets.

15. The method of claim 1, wherein the freeze-dried platelet-derived material further comprises a polysugar, wherein the polysugar is polysucrose, wherein the pH of the freeze-dried platelet-derived material is in the range of pH 6.2 to pH 7.8, and wherein the freeze-dried platelet-derived material is a rehydrated composition of freeze-dried platelets.

16. The method of claim 1, wherein the freeze-dried platelet-derived material is in the form of a powder, a liquid, a paste, a gel, or within a matrix.

17. The method of claim 3, wherein the administering is performed prior to surgery.

18. The method of claim 3, wherein administering is by infusion, wherein the freeze-dried platelet-derived material further comprises a polysugar, wherein the sugar is trehalose at a concentration of 10 mM to 500 mM, wherein the polysugar is polysucrose, and wherein the pH of the rehydrated freeze-dried platelet-derived material is in the range of pH 6.2 to pH 7.8.

19. The method of claim 1, wherein the freeze-dried platelet-derived material is in the form of a powder.

20. The method of claim 3, wherein the subject has been administered clopidogrel or ticlopidine, and the clopidogrel or the ticlopidine is present in the subject at the time of administering.
